(19) United States
(12) Patent Application Publication
(10) Pub. No.: US 2016/0262707 A1
(43) Pub. Date: Sep. 15, 2016

(54) PORTABLE DEVICES AND METHODS FOR MEASURING NUTRITIONAL INTAKE

(71) Applicant: BLACKTREE FITNESS TECHNOLOGIES INC., Kitchener (CA)

(72) Inventor: Emmanuel Jesse DeVries, Kitchener (CA)

(21) Appl. No.: 15/032,437

(22) PCT Filed: Oct. 24, 2014
(86) PCT No.: PCT/CA2014/000767

§ 371 Date: Apr. 27, 2016

Related U.S. Application Data
(60) Provisional application No. 61/896,114, filed on Oct. 27, 2013.

(51) Int. Cl. A61B 5/00; A61M 5/172; A61B 5/024; A61B 5/145; A61B 5/1455

(52) U.S. Cl. CPC A61B 5/7282; A61B 5/6802; A61B 5/14532; A61B 5/1455; A61B 5/4839; A61B 5/742; A61B 5/02416; A61M 5/1723; A61M 2230/201

(57) ABSTRACT

The present specification includes, amongst other things, a portable monitoring device to calculate caloric intake, the monitoring device comprising (i) a housing, such as a bracelet, having a physical size and shape that is wearable on the human body, (ii) a blood glucose sensor, disposed in the housing, to generate data which is representative of the blood glucose concentration of the user, (iii) a blood triglycerides sensor, disposed in the housing, to generate data which is representative of the blood triglycerides concentration of the user, and (iv) processing circuitry, disposed in the housing and coupled to the blood glucose sensor and/or blood triglycerides sensor, to calculate caloric intake using data representative of the blood glucose concentration and/or blood triglyceride concentration of the user.

### PRIORITY CLAIM

The present specification claims priority to U.S. Provisional Patent Application 61/896,114 filed Oct. 27, 2013, the contents of which are incorporated herein by reference.

### FIELD

The present specification relates generally to biosensors and nutritional science, and more particularly relates to various portable devices and methods for measuring nutritional intake.

### BACKGROUND

With the maturation of mobile and other computing technology, technological advances are evolving towards extending mobile technology through the use of wearable biosensors that connect to computers. In general theory, the biosensors collect biological data and in turn that data is fed to a computer which is programmed to compile and interpret that data. In turn those computing results can be used influence behavioural changes, such as changes to diet, exercise and the like. Such computing results could also be used to create a biofeedback device that, for example, automatically administers medications.

Enormous challenges are faced in developing such technology due to the variability between individual human physiologies and behaviours. From this perspective it can be viewed as a highly complex stochastic problem to develop such technology that produces meaningful results in a repeatable manner.

### SUMMARY

An aspect of this specification provides a device for monitoring nutritional intake comprising: at least one biosensor for receiving at least one of blood glucose data, blood triglyceride data, and, other nutrition-related physiological data when proximate to a blood vessel; a processing circuit connected to the biosensor output for calculating a nutritional measurement including at least a time representing a beginning of ingesting of food and a caloric intake after the time; an output device connected to the processing circuit for outputting at least one of the time and the caloric intake.

The output device can be further connected to an insulin pump having a control circuit; the control circuit being configured to meter a dose of insulin based on the nutritional measurement.

The output device can comprise a display configured to generate the nutritional measurement.

The output device can comprise a transmitter circuit for sending the nutritional measurement or the biosensor output data to an external processing circuitry.

The processing circuit can be further configured to calculate, as part of the nutritional measurement, at least one of a mass of carbohydrates intake, a mass of protein intake, a mass of fat intake, a glycemic index, and a glycemic load.

The biosensor can be a photoplethysmography sensor.

Methods implementing any of the foregoing are also contemplated.

### BRIEF DESCRIPTION OF THE DRAWINGS

Various embodiments according to the present specification will now be explained, by way of example only, in reference to the attached Figures in which:

FIG. 1 is a block diagram representation of an exemplary portable monitoring device, according to an embodiment of the present invention;

FIG. 2 is a block diagram representation of an exemplary portable monitoring device, according to an embodiment of the present invention;

FIG. 3 is a block diagram representation of an exemplary portable monitoring device, according to an embodiment of the present invention;

FIG. 4 is a block diagram representation of an exemplary portable monitoring device, according to an embodiment of the present invention;

FIG. 5 is a block diagram representation of an exemplary portable monitoring device, according to an embodiment of the present invention;

FIG. 6 is a block diagram representation of an exemplary portable monitoring device, according to an embodiment of the present invention;

FIG. 7 is a block diagram representation of an exemplary portable monitoring device, according to an embodiment of the present invention;

FIG. 8 is a block diagram representation of processing circuitry to calculate the caloric intake of the user based on sensor data;

FIG. 9 is a block diagram representation of an exemplary blood glucose sensor 52 which can be incorporated into any of the portable monitoring devices of FIGS. 1-7;

FIG. 10 is a block diagram representation of an exemplary blood triglycerides sensor 54 which can be incorporated into any of the portable monitoring devices of FIGS. 1-7;

FIG. 11 is a block diagram representation of an exemplary combined blood triglycerides sensor and blood glucose sensor which can be incorporated into any of the portable monitoring devices of FIGS. 1-7;

FIG. 12 is a flowchart representing an exemplary process of calculating caloric intake based on certain sensor data, according to an embodiment of the present invention;

FIG. 13 is a flowchart representing another exemplary process of calculating caloric intake based on certain sensor data, according to an embodiment of the present invention;

FIG. 14 is a graph illustrating an exemplary calculation of the Incremental Area Under the Curve of the blood glucose concentration data with respect to time;

FIG. 15 is a graph illustrating an exemplary calculation of the Incremental Area Under the Curve of the blood triglycerides concentration data with respect to time;

FIG. 16 is a flowchart representing an exemplary process of calculating mass of carbohydrates intake of the user based on certain sensor data, according to certain embodiments of the present invention;

FIG. 17 is a flowchart representing an exemplary process of calculating mass of carbohydrates intake of the user based on certain sensor data, according to certain embodiments of the present invention;

FIG. 18 is a graph illustrating an exemplary blood glucose concentration curve for starch-like carbohydrates;

FIG. 19 is a graph illustrating an exemplary blood glucose concentration curve for sugar-like carbohydrates;

FIG. 20 is a graph illustrating an exemplary target signal configured to isolate the effects of proteins intake on the blood glucose concentration data;

FIG. 21 is a graph illustrating an exemplary target signal configured to isolate the effects of fats intake on the blood glucose concentration data;

FIG. 22 is a block diagram representation of an exemplary portable monitoring device, according to an embodiment of the present invention;

FIG. 23 is a plot of typical data generated by the preferred embodiment, where the data was generated for a single meal for a single user. The measured caloric intake (from the area under the curve) was 162 calories, while the actual caloric intake, according to the packaged nutrition label, was 170 calories.

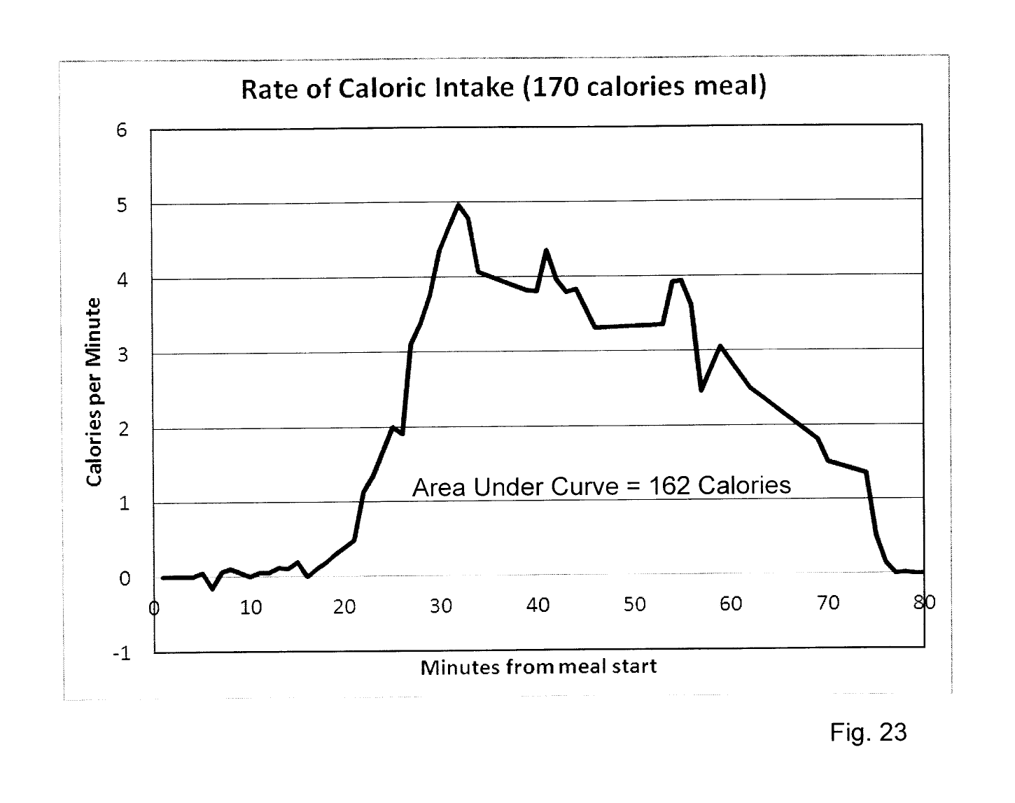

FIG. 24 is a plot of typical data generated by the preferred embodiment, where data was generated for all the meals in a particular day for a single user. The measured caloric intake (from the Incremental Area Under the Curve) is labelled for each meal on the corresponding region of the plot.

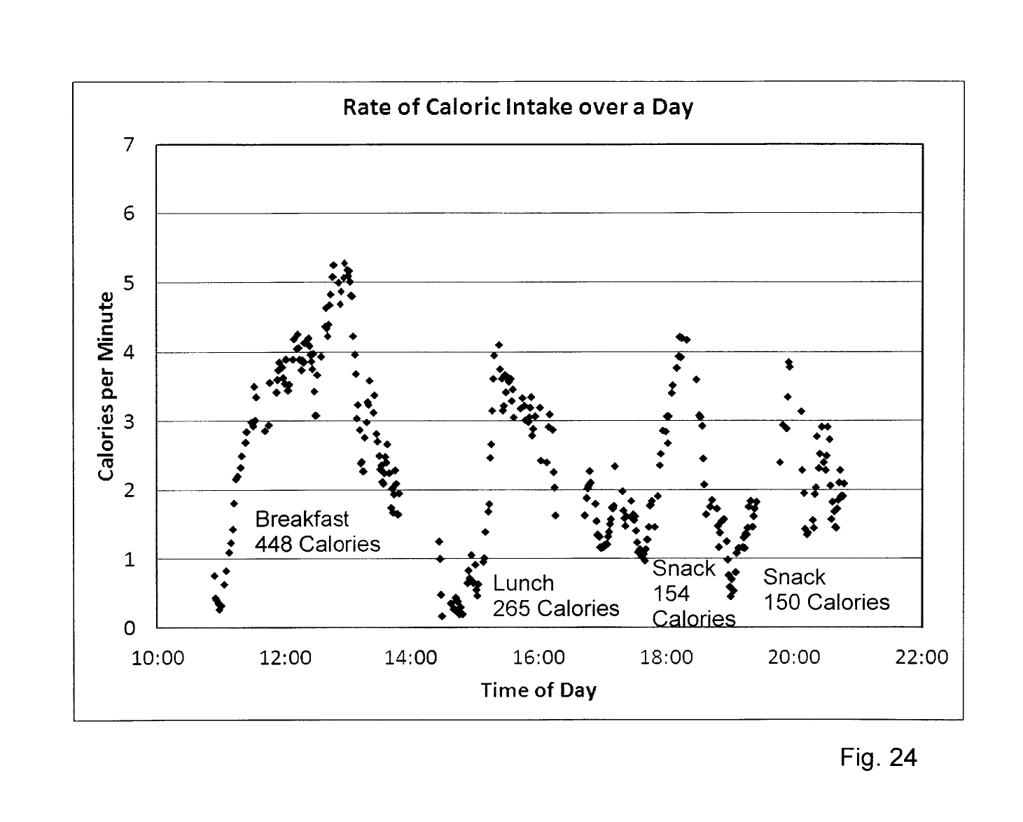

FIG. 25 is a side perspective view of an exemplary physical configuration of a portable monitoring device according to an embodiment;

FIG. 26 is a top perspective view of an exemplary physical configuration of a portable monitoring device according to an embodiment;

FIG. 27 is a block diagram representation of exemplary portable monitoring devices, according to an embodiment of the present invention;

FIG. 28 is a block diagram representation of exemplary portable monitoring devices, according to an embodiment of the present invention;

FIG. 29 is a block diagram representation of exemplary portable monitoring devices, according to an embodiment of the present invention;

FIG. 30 is a block diagram representation of exemplary portable monitoring devices, according to an embodiment of the present invention;

FIG. 31 is a block diagram representation of exemplary portable monitoring devices, according to an embodiment of the present invention.

### DETAILED DESCRIPTION OF THE EMBODIMENTS

The present specification is directed to portable monitoring devices, and methods of operating and controlling same, which monitor and calculate caloric intake due to the ingestion of food. The portable monitoring devices can comprise at least one of a blood glucose sensor and a blood triglycerides sensor, as well as processing circuitry configured to calculate caloric intake and/or other nutrition-related metrics.

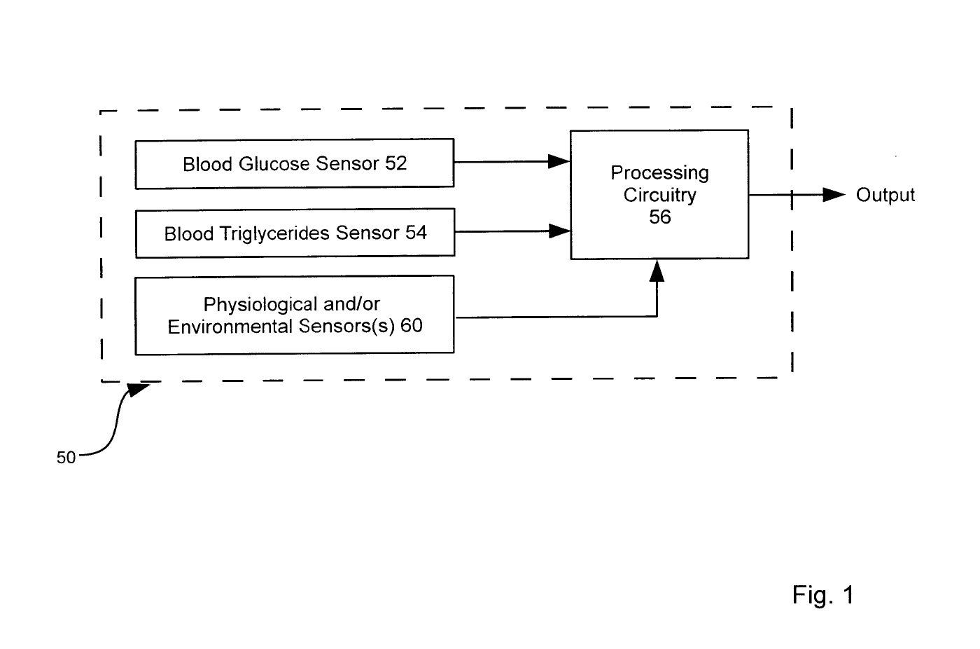

Referring now to FIG. 1, in a first, presently preferred embodiment there is provided a portable monitoring device 50 comprising a blood glucose sensor 52, a blood triglyceride sensor 54, and a physiological sensor 60, all of which generate outputs that are fed as inputs into a processing circuitry 56.

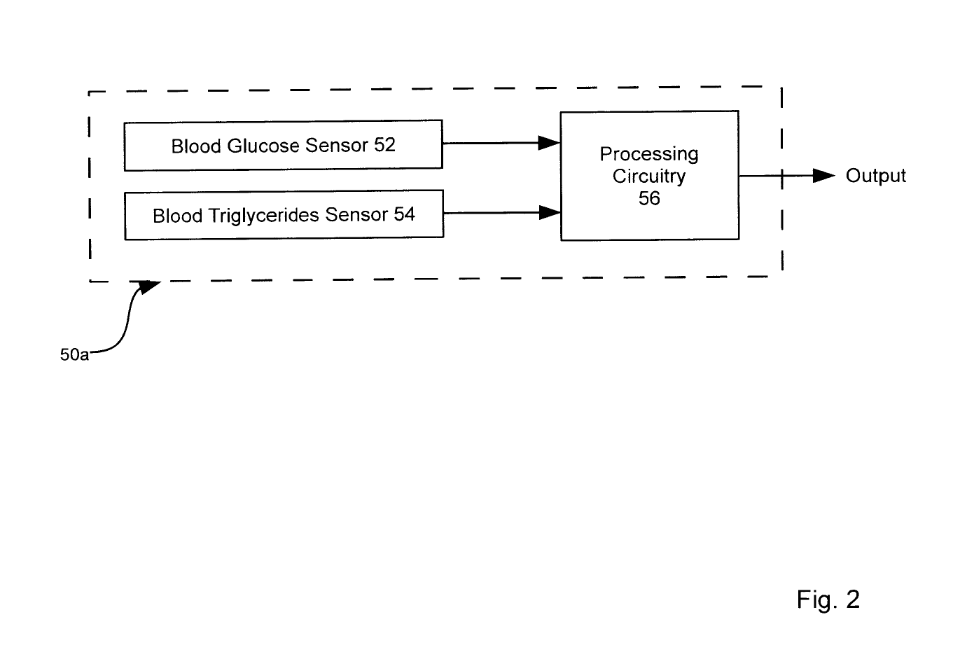

Referring now to FIG. 2, in a second embodiment there is provided a portable monitoring device 50a, (which is a variation on device 50) comprising a blood glucose sensor 52 and a blood triglyceride sensor 54, which generate outputs that are fed as inputs into a processing circuitry 56.

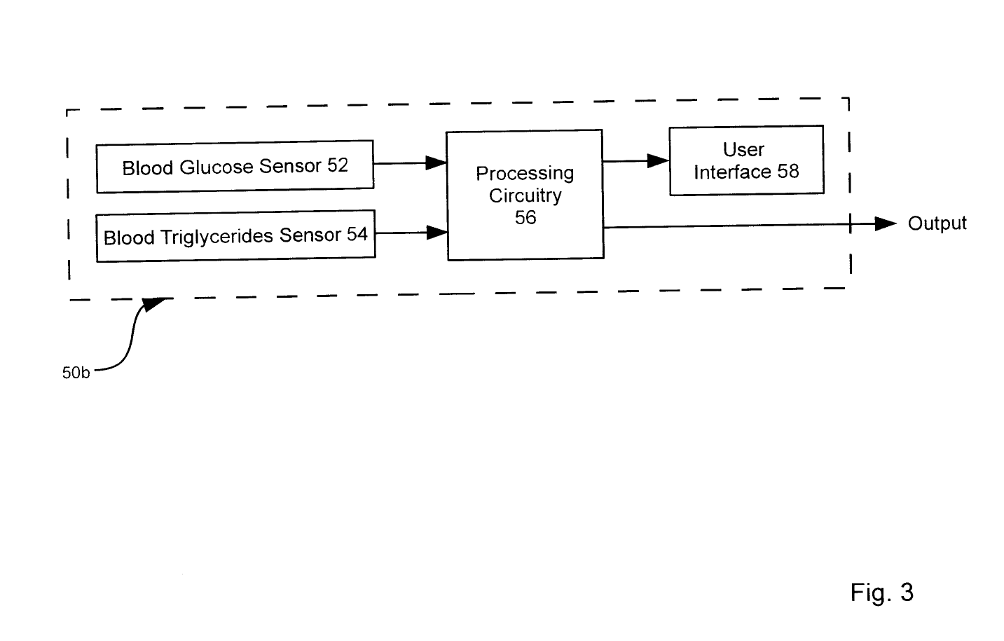

Referring now to FIG. 3, in a third embodiment there is provided a portable monitoring device 50b, (which is a variation on device 50) comprising a blood glucose sensor 52 and a blood triglyceride sensor 54, which generate outputs that are fed as inputs into a processing circuitry 56. In this embodiment, a user interface 58 can make information received from the processing circuitry 56 available to the user, and can make information received from the user available to the processing circuitry 56.

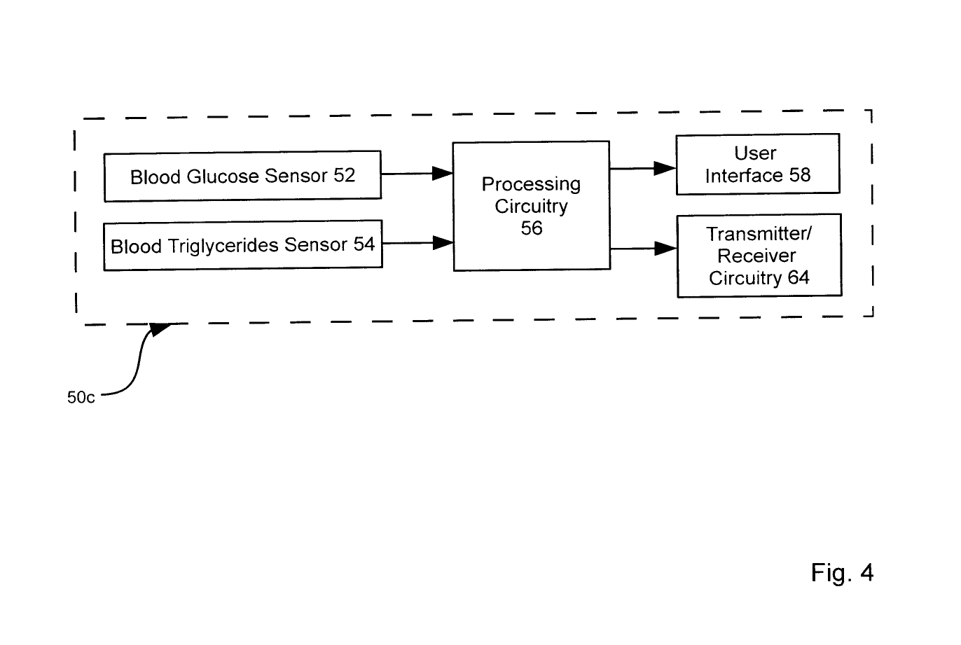

Referring now to FIG. 4, in a fourth embodiment there is provided a portable monitoring device 50c, (which is a variation on device 50) comprising a blood glucose sensor 52 and a blood triglyceride sensor 54, which generate outputs that are fed as inputs into a processing circuitry 56. In this embodiment, a user interface 58 can make information received from the processing circuitry 56 available to the user, and can make information received from the user available to the processing circuitry 56, and transmitter and/or receiver circuitry 64 can transmit information received from the processing circuitry 56 to an external device, and can receive information from an external device and make the information received the external device available to the processing circuitry 56.

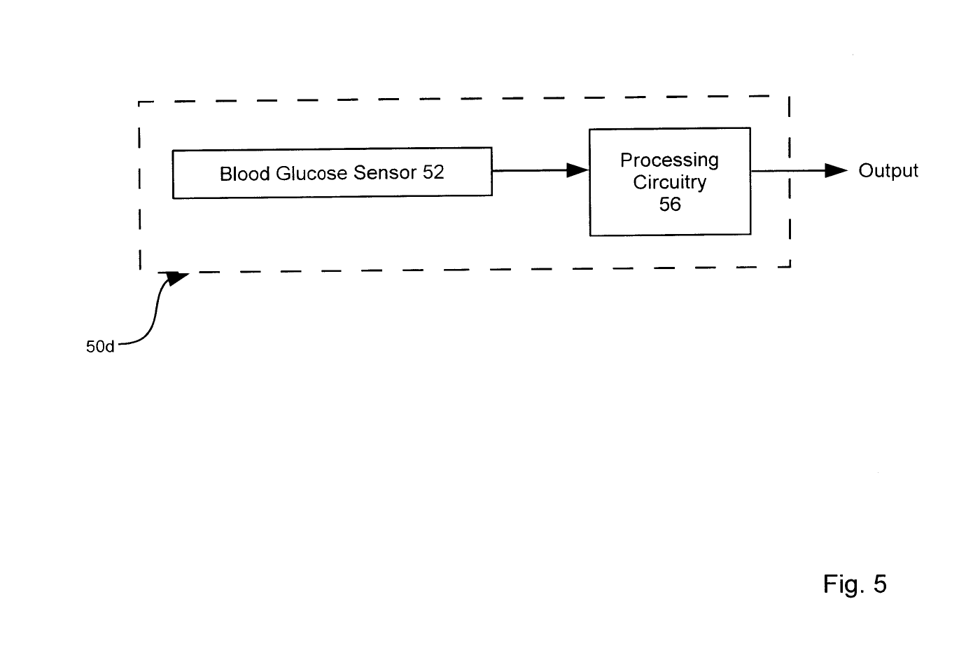

Referring now to FIG. 5, in a fifth embodiment there is provided a portable monitoring device 50d, (which is a variation on device 50) comprising a blood glucose sensor 52 which generate outputs that are fed as inputs into a processing circuitry 56.

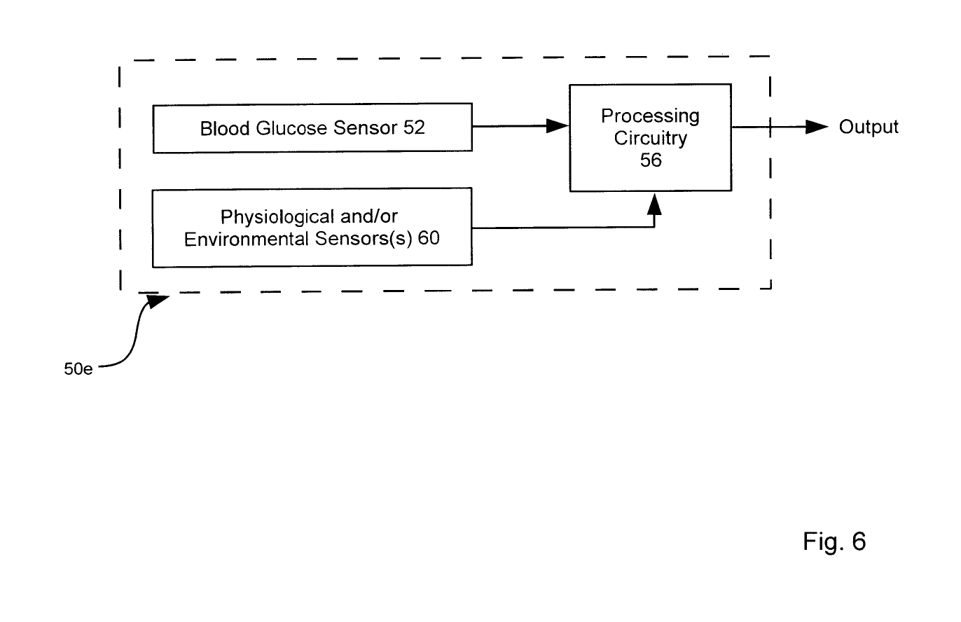

Referring now to FIG. 6, in a sixth embodiment there is provided a portable monitoring device 50e, (which is a variation on device 50) comprising a blood glucose sensor 52 and a physiological sensor 60, which generate outputs that are fed as inputs into a processing circuitry 56. Physiological sensor 60 will be discussed in greater detail below.

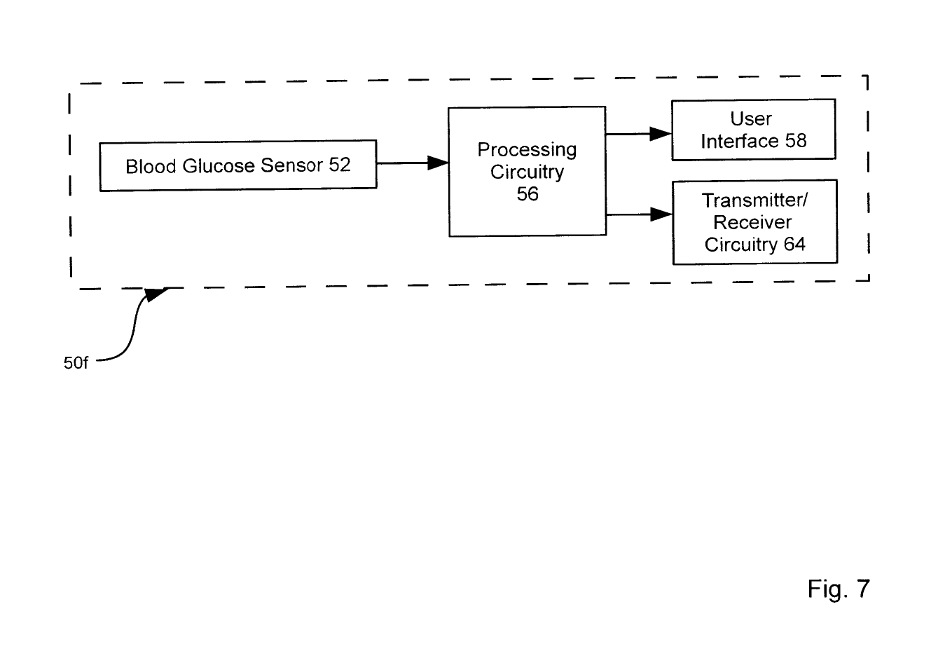

Referring now to FIG. 7, in a seventh embodiment there is provided a portable monitoring device 50f, (which is a variation on device 50) comprising a blood glucose sensor 52 which generate outputs that are fed as inputs into a processing circuitry 56. In this embodiment, a user interface 58 can make information received from the processing circuitry 56 available to the user, and user interface 58 can make information received from the user available to the processing circuitry 56. Furthermore, transmitter and/or receiver circuitry 64 can transmit information received from the processing circuitry 56 to an external device, and transmitter and/or receiver circuitry 64 can receive information from an external device and make the information received from an external device available to the processing circuitry 56. Examples of the external device will be discussed in greater detail below.

A person skilled in the art will now appreciate that further variations on the configurations shown in FIG. 1 through FIG. 7 are contemplated. Hereafter, portable monitoring device 50 will be discussed, but it is to be understood that all of the variations on portable monitoring device 50 (i.e. device 50, device 50a, device 50b . . . device 50g) can be applied to the following discussions according to the context of the following discussions.

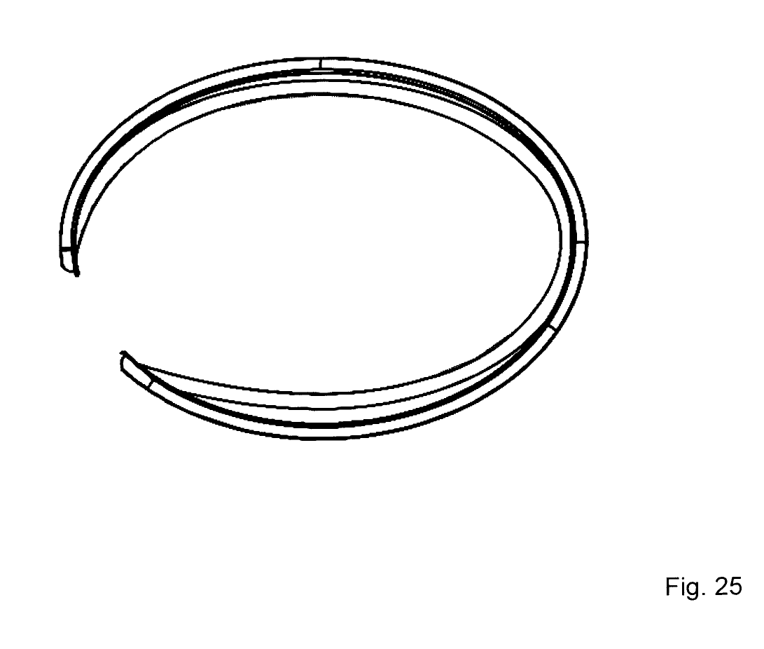

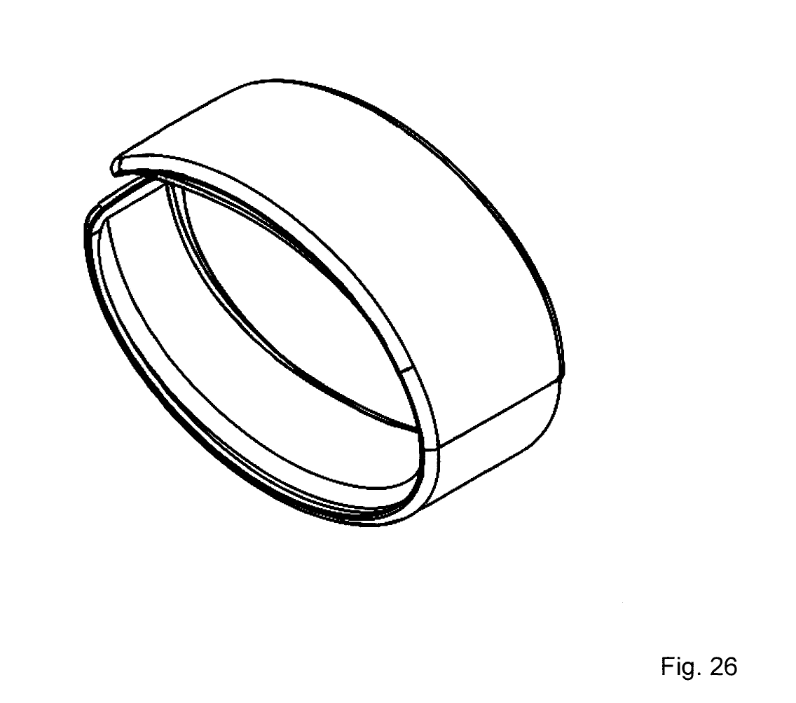

It is contemplated that at least a portion of the portable monitoring device 50 (including the one or more blood glucose sensors and/or blood triglycerides sensors) is worn, or affixed, during operation wherein the housing of the device includes a physical size and shape that facilitates coupling the body of the user. For example, the portable monitoring device 50 can be a bracelet worn on arm, wrist, ankle, waist, chest, and/or foot. It is presently preferred that the form factor of the portable monitoring device allows performance of normal or typical activities without undue hindrance. The portable monitoring device can include a mechanism (for example, a clip, strap, band and/or tie) for coupling or affixing the device to the body. An example bracelet configuration is shown in FIG. 25 and FIG. 26.

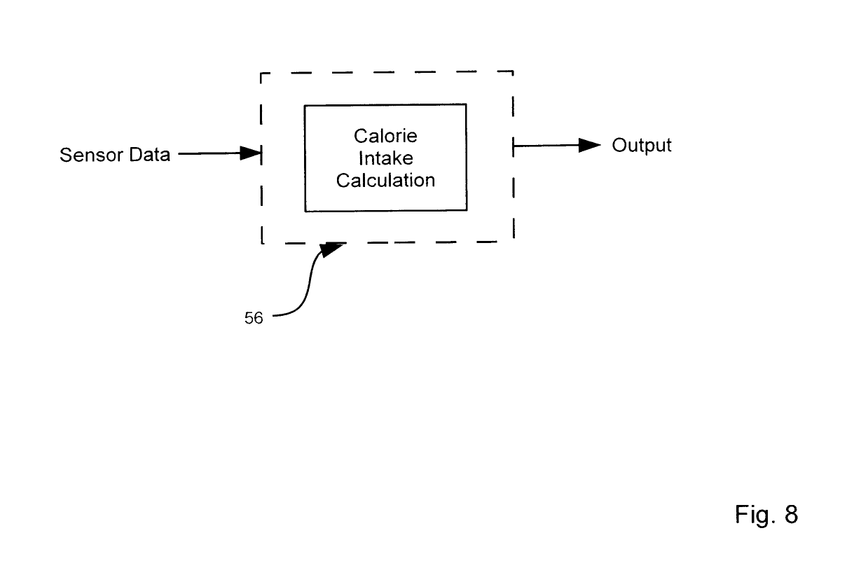

During operation, the blood glucose sensor 52 generates data which is representative of the blood glucose concentration of the user. The blood triglycerides sensor 54 generates data which is representative of the blood triglycerides concentration of the user. As shown in FIG. 8, the processing circuitry 56, using (i) data which is representative of the blood glucose concentration; and/or (ii) data which is representative of the blood triglycerides concentration of the user; calculates energy and/or caloric intake of the user.

Explained in greater detail, the processing circuitry 56 can be configured to calculate other nutrition-related metrics. Other nutrition-related metrics can include for example, (a) calories categorized into the macronutrient type (for example, carbohydrates, proteins, and fats), (b) the equivalent mass for a macronutrient type (for example, mass of carbohydrates, mass of proteins, and mass of fats), (c) a further breakdown for carbohydrates (for example, starches, sugars; or bread-like starches, pasta-like starches, glucose-like sugars, fructose-like sugars), (d) a further breakdown for proteins (for example, animal-based proteins, plant-based proteins), (e) a further breakdown for fats (for example, saturated fats, unsaturated fats), (f) the glycemic index, (g) the glycemic load. The examples in the preceding sentence can be broken into categories pertaining to a given period of time (for example, the past day, or a given week) or categories pertaining to each distinct meal, for example. Means for calculating these metrics will be discussed in greater detail below. This list of nutrition-related metrics (if applicable to the particular embodiment) is merely exemplary and is not intended to be exhaustive or limiting of the invention to, for example, the precise forms, techniques, flow, and/or configurations disclosed.

The processing circuitry 56 (or any other processing circuitry, such as the blood glucose processing circuitry 68 or blood triglycerides processing circuitry 70 described below) can be discrete or integrated logic, and/or one or more state machines, processors (suitably programmed) and/or field-programmable gate arrays (or combinations thereof); indeed any circuitry now known or later developed can be employed to calculate the energy and/or caloric intake of the user based on sensor data. In operation, the processing circuitry can perform or execute one or more applications, routines, programs and/or data structures that implement particular methods, techniques, tasks or operations described and/or illustrated herein. The functionality of the applications, routines, or programs can be combined or distributed. Further, the applications, routines or programs can be implemented by the processing circuitry using any programming language whether now known or later developed, including, for example, assembly, FORTRAN, C, C++, and BASIC, whether compiled or uncompiled code; all of which are intended to fall within the scope of the present invention.

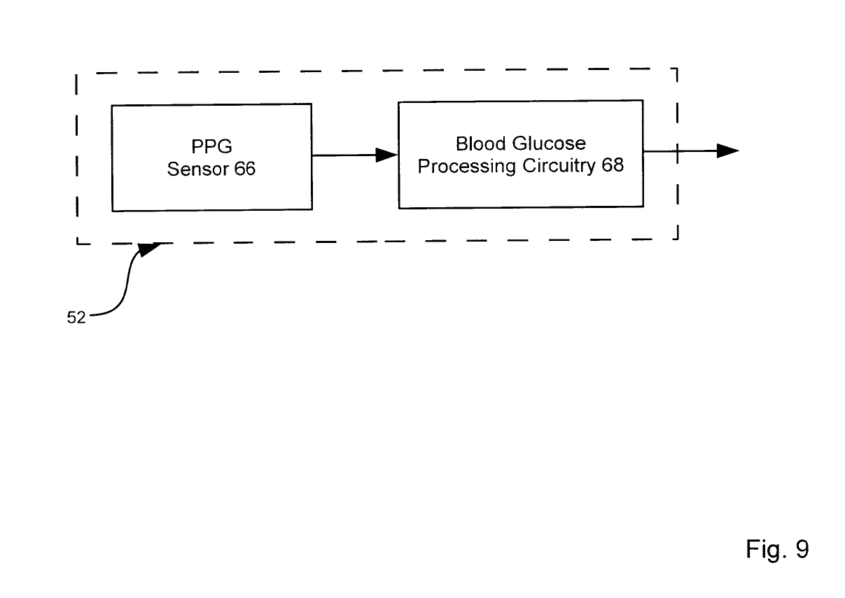

With reference to FIG. 9, in one embodiment, the blood glucose sensor 52 can include a photoplethysmography (PPG) sensor 66 and blood glucose processing circuitry 68 to assess the character of the photoplethysmography signal and calculate the blood glucose concentration. For example, the blood glucose processing circuitry can be configured according to the method described in the paper, “Non-invasive estimate of blood glucose and blood pressure from a photoplethysmograph by means of machine learning techniques”, by Enric Monte-Moreno and in U.S. patent application Ser. No. 13/128,205, the contents of which are incorporated herein by reference. In this embodiment, aspects of the photoplethysmography signal (defined as “variables” in a “feature vector”) that are related to the physiological response to blood glucose can be used to predict blood glucose concentrations by the use of a function estimation system. As a more particular example, the method mentioned in the previous sentence can be implemented with a function estimation system based on artificial neural networks (Haykin, 1998). As an additional example, the photoplethysmography sensor 66 can provide multiple photoplethysmography signals corresponding to different wavelengths of light. In another embodiment, an interstitial glucose sensor that generates data representative of the interstitial glucose concentration (Kulcu et al., 2003) can be substituted for the blood glucose sensor 52. Indeed, a variety of different types of sensors and sensing techniques, whether now known or later developed, that generate data which is representative of blood glucose concentration or interstitial glucose concentration are intended to fall within the scope of the present invention.

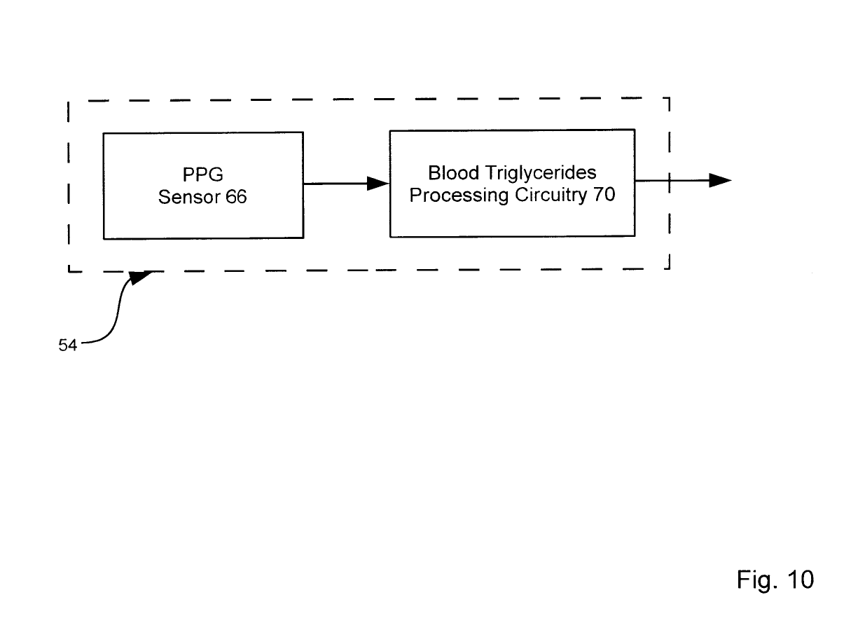

With reference to physiological sensor 60 in FIG. 1 and FIG. 6, the principle of estimating/measuring a metric (such as blood glucose concentrations) from the physiology measured by the photoplethysmography sensor can be generalized to any nutrition-related physiological metric that is related to desired output of nutrition-related metrics (calories per meal or time period, macronutrient breakdown, etc.). For example, in one embodiment with reference to FIG. 10, the blood triglycerides sensor 54 can include a photoplethysmography sensor 66 and blood triglycerides processing circuitry 70 to assess the photoplethysmography signal and calculate the blood triglycerides concentration, where the blood triglycerides processing circuitry 70 can be configured according to the method described in the paper, “Non-invasive estimate of blood glucose and blood pressure from a photoplethysmograph by means of machine learning techniques”, by Enric Monte-Moreno and in U.S. patent application Ser. No. 13/128,205, but with blood triglycerides concentration substituted for blood glucose concentration as the estimated/measured metric. As an additional example, the photoplethysmography sensor 66 can provide multiple photoplethysmography signals corresponding to different wavelengths of light. In another embodiment, an interstitial triglycerides sensor that generates data representative of the interstitial triglycerides concentration (Parini et al., 2006) can be substituted for the blood triglycerides sensor 54. Indeed, a variety of different types of sensors and sensing techniques, whether now known or later developed, that generate data which is representative of blood triglycerides concentration or interstitial triglycerides concentration are intended to fall within the scope of the present invention.

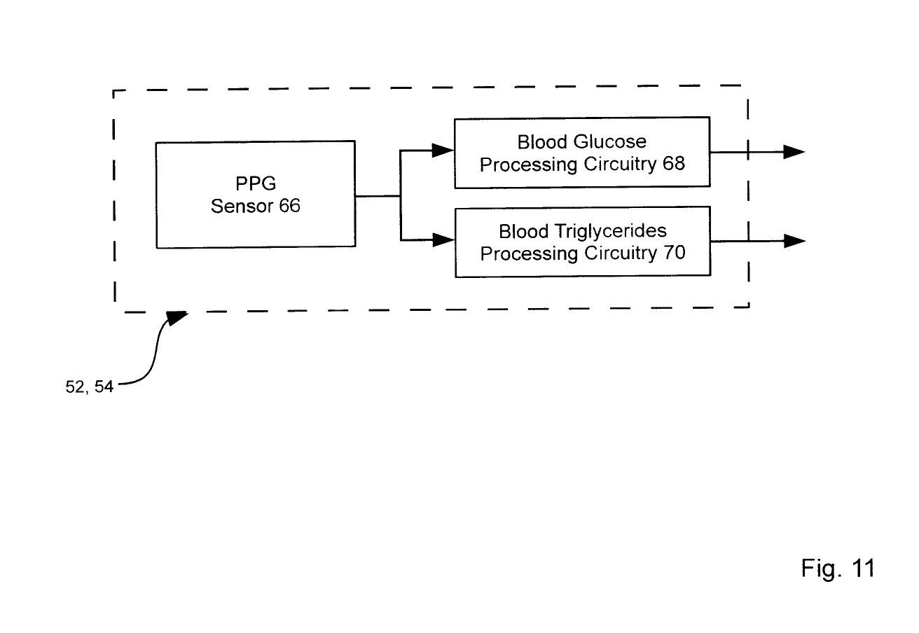

With reference to FIG. 11, in one embodiment the blood glucose sensor 52 and blood triglycerides sensor 54 can be implemented as per FIG. 9 and FIG. 10 but using a single photoplethysmography sensor 66 in common.

As mentioned above, the processing circuitry 56 employs (i) data which is representative of the blood glucose concentration and/or (ii) data which is representative of the blood triglycerides concentration of the user, and calculates energy and/or caloric intake of the user. For example, the blood glucose data can be in the form of blood glucose concentration(s) for a given time, either continuous with respect to time or sampled at specific times (for example, sampled about every 5 minutes, or sampled whenever a high quality signal is likely to be present, based on a signal quality estimation circuit or application). As a further example, the blood glucose data can be limited to a period of time, for example over the last about 1 hour to about 4 hours. For example, the blood triglycerides data can be in the form of blood triglycerides concentration(s) for a given time, either continuous with respect to time or sampled at specific times (for example, sampled about every 5 minutes, or sampled whenever a high quality signal is likely to be present, based on a signal quality estimation circuit or application). As a further example, the blood triglycerides data can be limited to a period of time, for example over the last about 1 hour to about 6 hours.

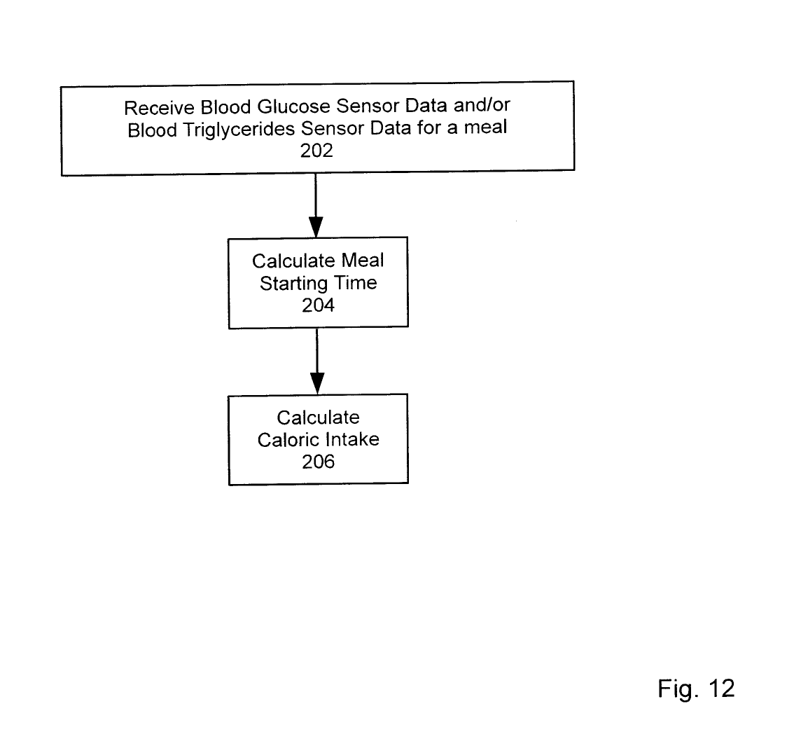

In one embodiment, the processing circuitry 56 implements a process based on the flowchart of FIG. 12. For example, with reference to FIG. 12, the processing circuitry 56 receives the blood glucose data (block 202), from which processing circuitry 56 calculates an estimate of the starting time for a given meal (block 204). For example, to calculate the starting time of a meal, a cross-correlation calculation can be performed between the blood glucose concentration signal and a target signal chosen to represent the temporal effects of a typical meal on the blood glucose concentration signal. The result of this calculation can be used in a threshold calculation (which evaluates to be “true” if the input is greater than (or alternatively, less than) an appropriate threshold, and evaluates false otherwise) in order to identify the meal start time. As another example, a stochastic estimator can be used to calculate the starting time for a given meal, using blood glucose concentration data over time as input features and the meal start time as predicted output. As another example, the starting time of a given meal can be manually entered by the user, to either augment or replace an automated calculation. For example, user manual entry of the starting time of a given meal can be used to calibrate the automated calculation of the starting time of a given meal. In this embodiment, using the meal start time, as well as blood glucose data and/or blood triglycerides data, the caloric intake can be calculated (block 206).

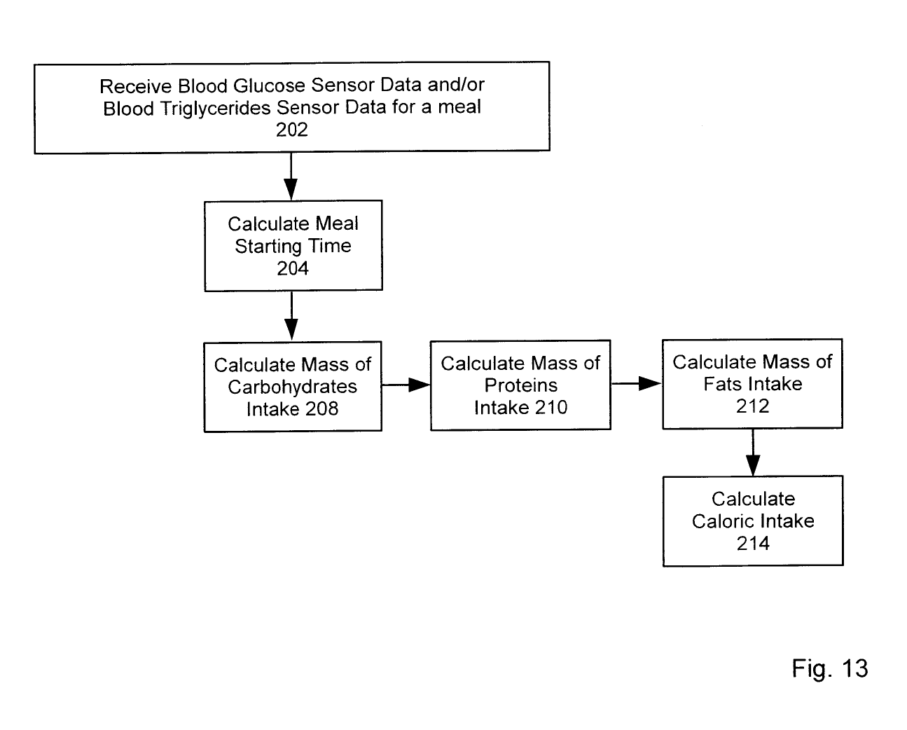

As a more specific example, with reference to FIG. 13, the processing circuitry 56 (i) uses the meal start time and blood glucose data to calculate the mass of carbohydrates intake (block 208), and/or (ii) uses the meal start time and blood glucose data to calculate the mass of proteins intake (block 210), and/or (iii) uses the meal start time, and blood glucose data and/or blood triglycerides data to calculate the mass of fats intake (block 212). The data representing (i) mass of carbohydrates intake, and/or (ii) mass of proteins intake, and/or (iii) mass of fats intake can then be evaluated in order to calculate the caloric intake of the user (block 214). (Note that a person skilled in the art will recognize that not all blocks need be performed in the exact order shown and that certain blocks can be performed in parallel according to the specific implementation. This note, according to the context, can be applicable to other flowcharts discussed herein.) In this regard, for example, the relationship can be expressed as:

Cᵢₙₜₐₖₑ=4*m_{carbohydrates}+4*mₚᵣₒₜₑᵢₙₛ+9*m_{fats}, (1)

where Cᵢₙₜₐₖₑ is the caloric intake [kcal], m_{carbohydrates} is the mass of carbohydrates intake [grams], mₚᵣₒₜₑᵢₙₛ m is the mass of proteins intake [grams], and m_{fats} is the mass of fats intake [grams].

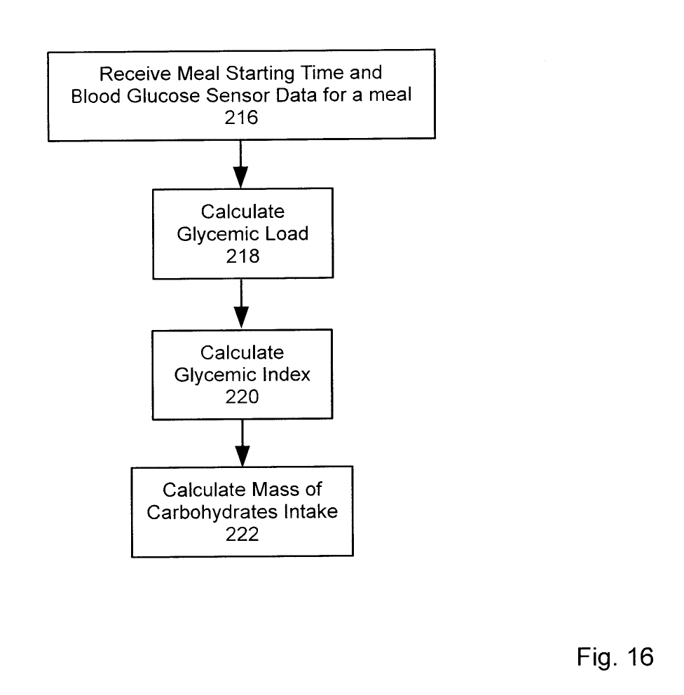

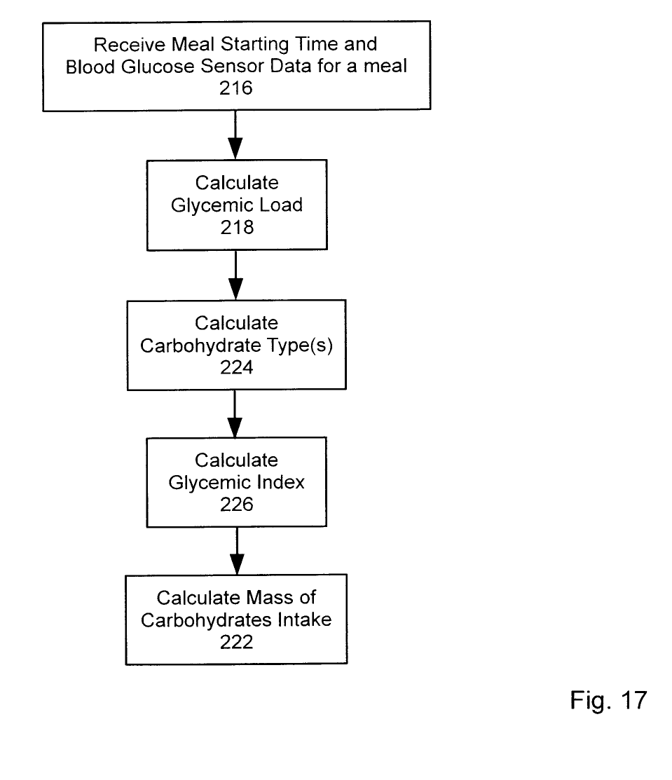

In one embodiment, with reference to block 208 of FIG. 13, block 222 of FIG. 16, and block 222 of FIG. 17, the processing circuitry 56 calculates the mass of carbohydrates intake according to the relationship expressed as:

m_{carbohydrates}=GI/GL*100, (2)

where m_{carbohydrates} is the mass of carbohydrates intake [grams], GL is the glycemic load, and GI is the glycemic index.

For example, with reference to block 218 of FIG. 16 and block 218 of FIG. 17, the GL value can be calculated according to the relationship expressed as:

GL=IAUC/IAUC_{1g}, (3)

where GL is the glycemic load, IAUC is the Incremental Area Under the Curve of the blood glucose concentration data, and IAUC_{1g} is the Incremental Area Under the Curve of the blood glucose concentration data due to intake of 1 gram of glucose (or equivalent) (Wolever et al., 2006).

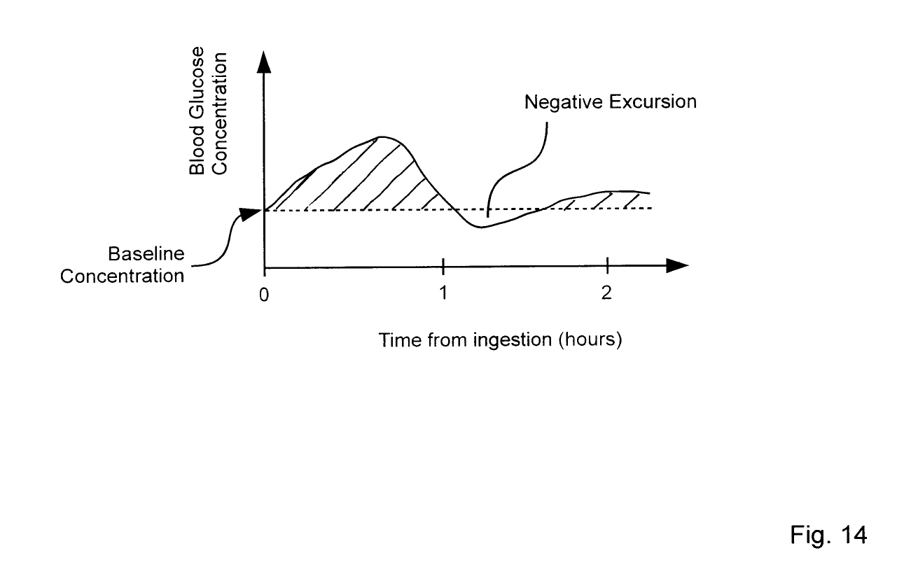

For example, with reference to FIG. 14, the IAUC can be calculated as the area under the curve of blood glucose concentration with respect to time, with the baseline (“pre-meal”) blood glucose concentration subtracted, for the about 2 hour period of time following the start of a meal, and with the negative excursions (relative to the baseline) excluded. (Other time periods can be used, for example about 1 hour-about 4 hours (Chlup et al., 2010).) However, any calculation for IAUC, whether now known or later developed, can be used and are intended to fall within the scope of the present invention. Additionally, in order to improve accuracy, the IAUC_{1g} value can be predetermined, or can be selected from pre-set values based on demographic information (for example, age, gender, height, and/or weight) (Moghaddam et al., 2006) and/or can be calibrated to the user, for example based on manual entry of nutritional information (e.g. calories and/or macronutrients) for a certain meal or meals (for example, one time or occasionally), and/or can be calibrated to the user by the use of additional physiological data (for example automatically measured/calculated and/or manually entered).

For example, referring to Equation (3), the employed IAUC value can first be adjusted based on a function according to:

IAUC′=f(IAUC), (4)

where IAUC′ is the resulting adjusted Incremental Area Under the Curve, f() is a suitable function (chosen in order to improve accuracy; for example, a polynomial, or more specifically a 1st order polynomial, a 2nd order polynomial, or a 3rd order polynomial), and IAUC is the original Incremental Area Under the Curve.

With reference to block 220 of FIG. 16, using the meal start time and blood glucose data, the processing circuitry 56 can calculate the GI for a given meal. As an example, the calculation of GI can be performed as a linear combination of the blood glucose concentrations sampled at specific times with respect to the start of a meal (for example, about every 5 minutes for the period of about 2 hours following the start of a meal). As another example, the calculation of GI can be implemented by a stochastic estimator (for example based on linear regression (Draper & Smith, 1998) (Rifkin & Lippert, 2007), artificial neural networks (Haykin, 1998), support vector machines (Chang & Lin, 2013), and/or random forests (Breiman, 2001)), where the input features are the blood glucose concentrations sampled at specific times with respect to the start of a meal.

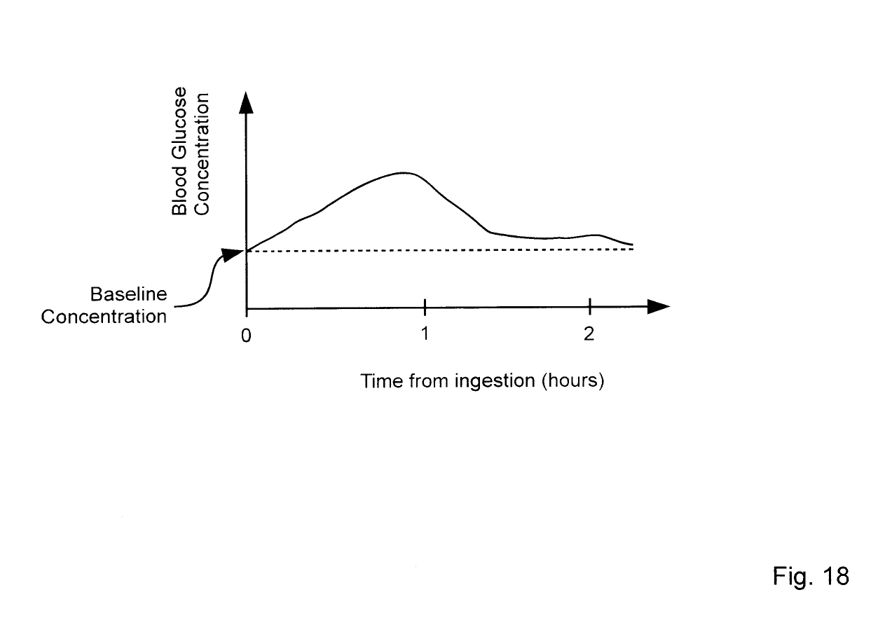

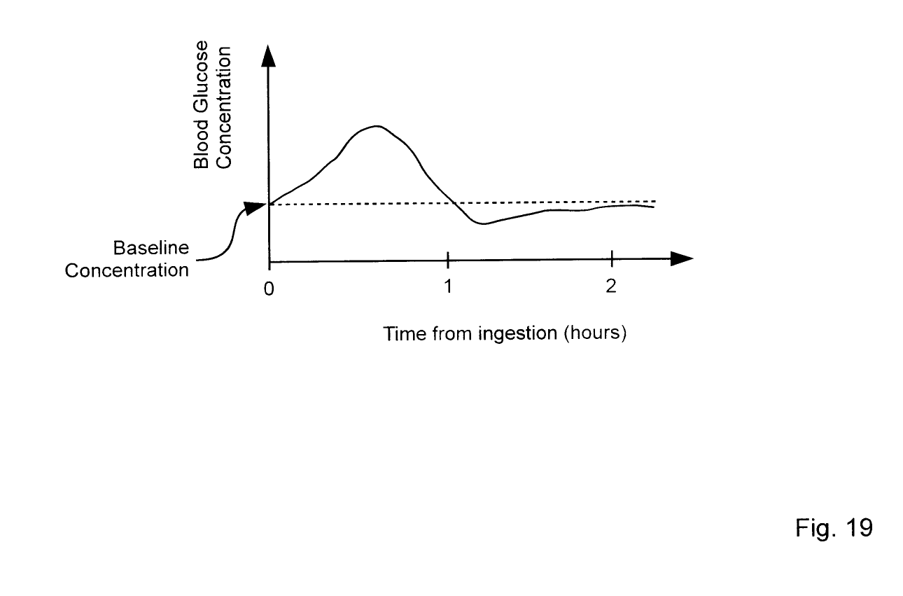

With reference to FIG. 17, using the meal start time and blood glucose data, the carbohydrates type(s) is/are calculated (block 224). For example, carbohydrates can be categorized as starch-like (See, FIG. 18), or sugar-like (See, FIG. 19). As a further example, sugar-like carbohydrates can be categorized as glucose-like or non-glucose-like, and starch-like carbohydrates can be categorized as bread-like and pasta-like. For exemplary data illustrating these relationships between the blood glucose data and carbohydrates categories, see (Brand-Miller et al., 2009). Again with reference to FIG. 17, this categorization of carbohydrate types can be employed in the calculation of GI (block 226), in order to improve accuracy. For example, the carbohydrate type(s) can be used to select from a set of linear combinations of the blood glucose concentrations sampled at specific times with respect to the start of a meal, each optimized for the respective carbohydrate type. As another example, the carbohydrate type(s) can be used to select from a set of stochastic estimators for calculating GI (with input features including the blood glucose concentrations sampled at specific times with respect to the start of a meal), each optimized for the respective carbohydrate type. In another exemplary embodiment, the aspect of processing circuitry 56 that calculates mass of carbohydrates intake can be implemented as some combination of the previously mentioned techniques and/or any other techniques that are intended to calculate mass of carbohydrates intake.

In one embodiment, with reference to block 210 of FIG. 13, the processing circuitry 56 calculates the mass of proteins intake from the blood glucose concentration signal by first calculating the cross-correlation of the blood glucose concentration signal and a target signal chosen to isolate the effects of protein intake on the blood glucose concentration signal. This calculation of the protein-correlated blood glucose signal can be expressed as:

blood_glucoseₚᵣₒₜₑᵢₙ[n]=(blood_glucose*protein_target)[n], (5)

where blood_glucoseₚᵣₒₜₑᵢₙ is the protein-correlated blood glucose signal, n is the time index, blood_glucose is the blood glucose concentration signal, protein_target is representative of the blood glucose concentration signal when protein is ingested in relative isolation, and * is the cross-correlation operator.

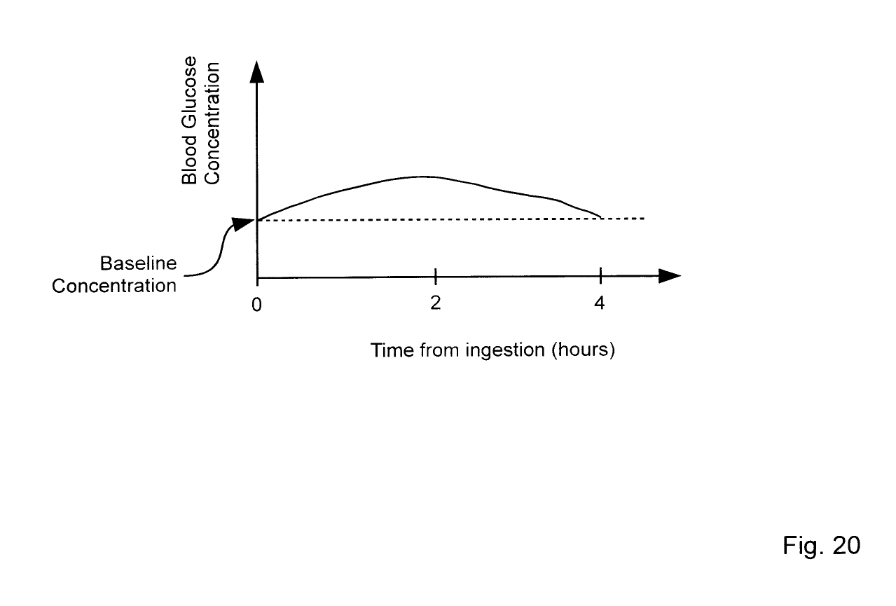

For an example signal for protein_target, see FIG. 20. In this example, protein_target is the blood glucose concentration signal due to a reference meal of protein in isolation i.e. with minimal carbohydrates, fats, or other non-protein nutrients.

Continuing in the present embodiment, the processing circuitry 56, given blood_glucoseₚᵣₒₜₑᵢₙ, can calculate the mass of proteins intake according to the relationship expressed as:

mₚᵣₒₜₑₙₛ=IAUC_{correlated}/IAUC_{1g}, (6)

where mₚᵣₒₜₑᵢₙₛ m is the mass of proteins intake [grams], IAUC_{correlated} is the Incremental Area Under the Curve of blood_glucoseₚᵣₒₜₑᵢₙ (from Equation (5)), and IAUC_{1g} is the Incremental Area Under the Curve of blood_glucoseₚᵣₒₜₑᵢₙ due to intake of 1 gram of protein.

For example, the Incremental Area Under the Curve (IAUC) used in Equation (6) is similar to the IAUC used in Equation (3), except that in Equation (6), the input is the correlated blood glucose signal, blood_glucoseₚᵣₒₜₑᵢₙ. Additionally, in order to improve accuracy, the IAUC_{1g} value can be predetermined, or can be selected from pre-set values based on demographic information (for example, age, gender, height, and/or weight) (Moghaddam et al., 2006) and/or can be calibrated to the user, for example based on some manual entry of nutritional information (e.g. calories and/or macronutrients) for some meal or meals (for example, one time or occasionally), and/or can be calibrated to the user by the use of additional physiological data (for example automatically measured/calculated and/or manually entered).

For example, referring to Equation (6), the employed IAUC_{correlated} value can first be adjusted based on a function according to:

IAUC_{correlated}′=f(IAUC_{correlated}), (7)

where IAUC_{correlated}′ is the resulting adjusted Incremental Area Under the Curve (of blood_glucoseₚᵣₒₜₑᵢₙ), f() is a suitable function (chosen in order to improve accuracy; for example, a polynomial, or more specifically a 1st order polynomial, a 2nd order polynomial, or a 3rd order polynomial), and IAUC_{correlated} is the original Incremental Area Under the Curve (of blood_glucoseₚᵣₒₜₑᵢₙ).

In another exemplary embodiment, the aspect of processing circuitry 56 that calculates the mass of proteins intake can be implemented by a stochastic estimator (for example based on linear regression (Draper & Smith, 1998) (Rifkin & Lippert, 2007), artificial neural networks (Haykin, 1998), support vector machines (Chang & Lin, 2013), and/or random forests (Breiman, 2001)), where the input features are the blood glucose concentrations sampled at specific times with respect to the start of a meal. In yet another exemplary embodiment, the aspect of processing circuitry 56 that calculates mass of proteins intake can be implemented as some combination of the previously mentioned techniques and/or any other techniques that calculate mass of proteins intake.

In lieu or in combination with the use of a blood glucose sensor in order to generate data which is representative of the user's blood glucose concentration, a blood protein sensor (not shown) can be used in order to generate data which is representative of blood protein concentration for any of the above techniques that calculate the mass of proteins intake.

Referring again to block 212 FIG. 13, the processing circuitry 56 calculates the mass of fats intake according to the relationship expressed as:

m_{fats}=IAUC/IAUC_{1g}, (8)

where m_{fats} is the mass of fats intake [grams], IAUC is the Incremental Area Under the Curve of the blood triglycerides concentration data, and IAUC_{1g} is the Incremental Area Under the Curve of the blood triglycerides concentration curve due to intake of 1 gram of fat (or equivalent).

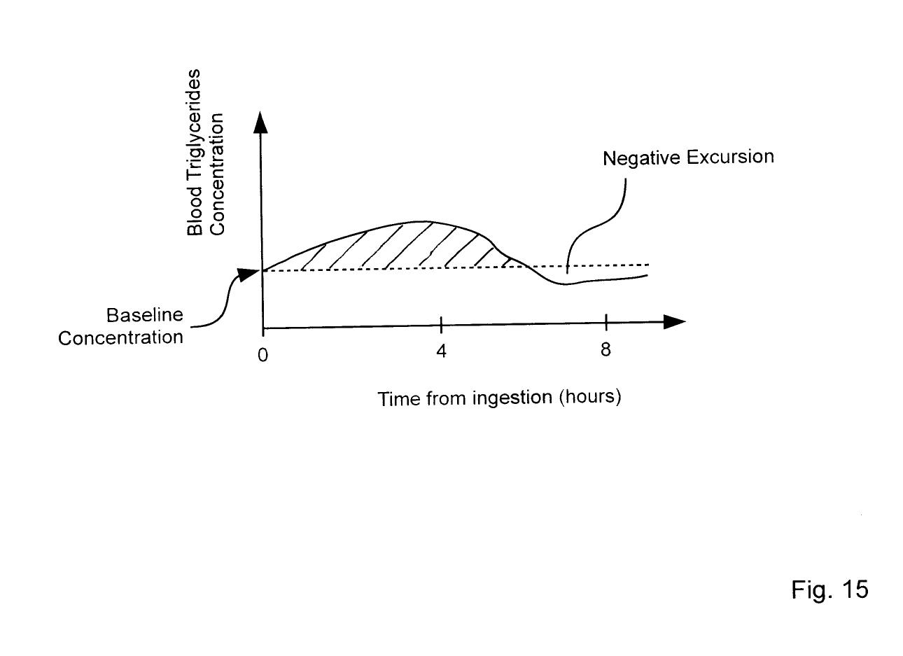

For example, the Incremental Area Under the Curve (IAUC) used in Equation (8) is similar to the IAUC used in Equation (3), except that in Equation (8), the input is the blood triglycerides concentration signal (For example, see FIG. 15). Additionally, in order to improve accuracy, the IAUC_{1g} value can be predetermined, or can be selected from pre-set values based on the user's demographic information (for example, age, gender, height, and/or weight) (Moghaddam et al., 2006) and/or can be calibrated to the user, for example based on some manual entry of nutritional information (e.g. calories and/or macronutrients) for some meal or meals (for example, one time or occasionally), and/or can be calibrated to the user by the use of additional physiological data (for example automatically measured/calculated and/or manually entered).

For example, referring to Equation (8), the employed IAUC value can first be adjusted based on a function according to:

IAUC′=f(IAUC), (9)

where IAUC′ is the resulting adjusted Incremental Area Under the Curve, f() is a suitable function (chosen in order to improve accuracy; for example, a polynomial, or more specifically a 1st order polynomial, a 2nd order polynomial, or a 3rd order polynomial), and IAUC is the original Incremental Area Under the Curve.

In one embodiment, with reference to block 212 of FIG. 13, the processing circuitry 56 calculates the mass of fats intake from the blood glucose concentration signal by first calculating the cross-correlation of the blood glucose concentration signal and a target signal chosen to isolate the effects of fats intake on the blood glucose concentration signal. This calculation of the fat-correlated blood glucose signal can be expressed as:

blood_glucose_{fat}[n]=(blood_glucose*fat_target)[n], (10)

where blood_glucose_{fat} is the fat-correlated blood glucose signal, n is the time index, blood_glucose is the blood glucose concentration signal, fat_target is representative of the blood glucose concentration signal when fat is ingested in relative isolation, and * is the cross-correlation operator.

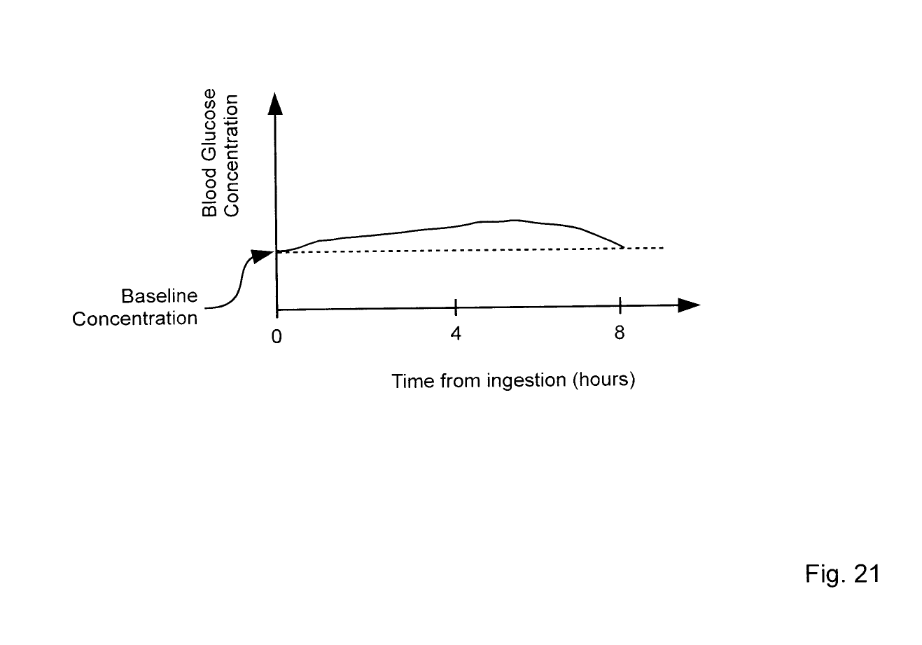

For an example signal for fat_target, see FIG. 21. In this example, fat_target is the blood glucose concentration signal due to a reference meal of fat in isolation i.e. with minimal carbohydrates, protein, or other non-fat nutrients.

Continuing in the present embodiment, the processing circuitry 56, given blood_glucose_{fat}, can calculate the mass of fats intake according to the relationship expressed as:

m_{fats}=IAUC_{correlated}/IAUC_{1g}, (11)

where m_{fats} is the mass of fats intake [grams], IAUC_{correlated} is the Incremental Area Under the Curve of blood_glucose_{fat} (from Equation (10)), and IAUC_{1g} is the Incremental Area Under the Curve of blood_glucose_{fat} due to intake of 1 gram of fat (or equivalent).

For example, referring to Equation (11), the employed IAUC_{correlated} value can first be adjusted based on a function according to:

IAUC_{correlated}′=f(IAUC_{correlated}), (12)

where IAUC_{correlated}′ is the resulting adjusted Incremental Area Under the Curve (of blood_glucose_{fat}), f() is a suitable function (chosen in order to improve accuracy; for example, a polynomial, or more specifically a 1st order polynomial, a 2nd order polynomial, or a 3rd order polynomial), and MUC_{correlated} is the original Incremental Area Under the Curve (of blood_glucose_{fat}).

In another exemplary embodiment, the aspect of processing circuitry 56 that calculates mass of fats intake can be implemented by a stochastic estimator (for example based on linear regression (Draper & Smith, 1998) (Rifkin & Lippert, 2007), artificial neural networks (Haykin, 1998), support vector machines (Chang & Lin, 2013), and/or random forests (Breiman, 2001)), where the input features are the blood glucose concentrations and/or blood triglyceride concentrations sampled at specific times with respect to the start of a meal. In yet another exemplary embodiment, the aspect of processing circuitry 56 that calculates mass of fats intake can be implemented as some combination of the previously mentioned techniques and/or any other techniques that are intended to calculate mass of fats intake.

In lieu or in combination with the use of blood triglycerides sensor 54 in order to generate data representative of the blood triglycerides concentration, a blood lipids sensor (not shown) can be used in order to generate data representative of the user's blood lipids concentration for any of the above techniques. For example, blood lipids can include blood cholesterol.

In one embodiment, the processing circuitry 56 is configured to calculate nutritional quality metric(s). For example, a nutritional quality metric can be implemented by the relationship expressed as:

Q=f(GI,GL,carbohydrates_categories,m_{carbohydrates},mₚᵣₒₜₑᵢₙₛ,m_{fats}), (13)

where Q is a quality metric, f() is a suitable function, GI is the glycemic index, GL is the glycemic load, carbohydrates_categories are representative of the categorization of the carbohydrates intake (described previously), m_{carbohydrates} is the mass of carbohydrates intake, mₚᵣₒₜₑᵢₙₛ is the mass of proteins intake, and m_{fats} is the mass of fats intake.

For example, the quality metric can be the glycemic index, expressed as:

Q=GI, (14)

where Q is a quality metric, and GI is the glycemic index.

As another example, the quality can be a function of GI and the breakdown of the macronutrients by caloric intake, expressed as:

Q=f(GI,C_{fats}/(C_{carbohydrates}+Cₚᵣₒₜₑᵢₙₛ+C_{fats})), (15)

where Q is a quality metric, f() is a suitable function, GI is the glycemic index, C_{fats} is the caloric intake due to fats, C_{carbohydrates} is the caloric intake due to carbohydrates, Cₚᵣₒₜₑᵢₙₛ is the caloric intake due to proteins.

In one embodiment, processing circuitry 56 implements techniques to account for the effects of another aspect or aspects of the user's physiology or environment (for example, physical exertion (O'Keefe et al., 2008), and/or stress levels, and/or circadian rhythm, and/or skin temperature, and/or environment temperature, and/or the quality of the previous night's sleep, and/or the time of day, and/or the environment light levels) in order to maximize accuracy when calculating caloric intake of the user and/or other nutritional metrics. For example, any or all of the techniques already mentioned can be augmented by taking the input of data representative of physical exertion (and/or other physiological or environmental variables) with respect to time. As a particular example, where a stochastic estimator is used, the physical exertion (and/or other physiological or environmental variables) data can be directed to additional feature(s) in the stochastic estimator (for example, as the physical exertion signal (and/or other physiological or environmental variables) sampled at specific times relative to the start of a given meal). As another particular example, the blood glucose concentration data (and/or blood triglycerides data) used in any of the techniques described herein can be adjusted according to the relationship expressed as:

bc_{adj}=bc*[s×M], (16)

where bc_{adj} is the adjusted blood concentration signal (for example, glucose or triglycerides), bc is the original blood concentration signal, s is the signal representative of another aspect of the user's physiology or environment (for example, physical exertion), M is a pre-set adjustment matrix, * is the element-wise product operator, and X is the matrix multiplication operator.

As an alternative example, the blood glucose concentration data and/or blood triglycerides data used in any techniques described herein can be adjusted according to multiple signals representative of other aspects of the user's physiology or the external environment. This can be implemented as additional features in the stochastic estimator(s), and/or according to the relationship expressed as:

bc_{adj}=bc*[s₁×M₁]+bc*[s₂×M₂]+ . . . +bc*[sₙ×Mₙ], (17)

where bc_{adj} is the adjusted blood concentration signal (for example, glucose or triglycerides), bc is the original blood concentration signal, sᵢ are the signals representative of other aspects of the user's physiology or environment (for example, physical exertion or time of day), Mᵢ are pre-set adjustment matrices, * is the element-wise product operator, and X is the matrix multiplication operator.

Referring now to FIG. 14, processing circuitry 56 can incorporate the calculated mass of proteins intake and/or mass of fats intake when calculating the mass of carbohydrates intake (block 208), in order to improve accuracy of the calculation (O'Keefe et al., 2008) (Petersen et al., 2009). Additionally, the processing circuitry 56 can incorporate the calculated mass of carbohydrates intake and/or mass of fats intake when calculating the mass of proteins intake (block 210), in order to improve accuracy of the calculation. Additionally, processing circuitry 56 can incorporate the calculated mass of carbohydrates intake and/or mass of proteins intake when calculating the mass of fats intake (block 212), in order to improve accuracy of the calculation. In the case of interdependencies in blocks 208, 210, and 212, an iterative approach can be used. Additionally, the processing circuitry 56 can incorporate other information about nutritional intake (for example, water intake, fiber intake, vitamins intake, minerals intake, phytochemicals intake) in order to improve the accuracy when calculating mass of carbohydrates intake, mass of proteins intake, and/or mass of fats intake. For example, the techniques of this paragraph can be implemented in combination with any other technique(s) described and/or illustrated herein.

In lieu or in combination with the use of sensors to generate data representative of the blood concentrations of certain nutrition-related physiological metrics or metabolites in the techniques described and/or illustrated herein (for example, glucose, triglycerides, cholesterol, lipids, and/or proteins), other nutrition-related physiological metrics can be used with the respective techniques and are intended to fall within the scope of the present invention. Additionally, the sensors are preferably non-invasive (not requiring penetration of the user's skin), but invasive sensors can be used. Additionally, data from sensors on an external device (for example, a mobile phone) can be used for the techniques in the present invention.

In one embodiment, the caloric intake calculations can be automatically calibrated by employing caloric expenditure data and making the assumption that (either for a given day or on average for a number of days):

Cᵢₙₜₐₖₑ=C_{expended}, (18)

where Cᵢₙₜₐₖₑ is the caloric intake, and C_{expenditure} is the caloric expenditure, e.g. due to metabolic activity such as exercise and the basal metabolism.

As an example, the prior technique can be augmented by employing data representative of the user's weight over a period of time (for example, a number of days), for example according to the relationship expressed as:

Cᵢₙₜₐₖₑ−C_{expenditure}=(M_{end}−Mₛₜₐᵣₜ)*K, (19)

where Cᵢₙₜₐₖₑ is the total caloric intake [kcal], C_{expenditure} is the total caloric expenditure [kcal], Mₛₜₐᵣₜ is the user's body mass at the start of the given period [lbs], M_{end} is the user's body mass at the end of the given period, and K is a constant (for example 3555 [kcal/lb]).

In one embodiment, the portable monitoring device 50 (or its variants) can track, in combination or in lieu of nutrition related metrics, other health related metrics. For example, the portable monitoring device can monitor and/or calculate caloric expenditure (for example, by the use of demographic information in addition to motion sensors and/or physiological sensors (for example, a heart rate sensor (for example, based on photoplethysmography or electrocardiography))). For example, portable monitoring device 50 can monitor and/or calculate sleep-related metrics of the user (for example, hours of sleep in given night, and/or hours of deep sleep), and/or provide for an alarm to wake the user at a selected time based on the user's circadian rhythm and pre-set time constraints. Portable monitoring device 50 can detect the sleep-related metrics based on a physiological and/or environmental sensors 60 (for example, a motion sensor, and/or a heart-rate sensor (for example, based on photoplethysmography, or electrocardiography), and/or a skin conductance sensor, and/or an electroencephalography sensor). For example, the portable monitoring device 50 can monitor and/or calculate stress-related metrics of the user based on data obtained by physiological and/or environmental sensor(s) 60. For example, portable monitoring device 50 can implement the stress-related metrics based on heart-rate variability derived from a heart-rate sensor (for example, based on photoplethysmography, or electrocardiography), and/or data from a skin conductance sensor, and/or data from an electroencephalography sensor.

In one embodiment, the portable monitoring device 50 can augment and/or replace calculations for nutrition-related metrics, for example using data from manual entry and/or photos of a given meal, and/or scans of a barcode or label on nutritional packaging, and/or pre-existing nutritional databases.

Device 50 can implement algorithms (such as the specific algorithms discussed above) in a real-time manner (for example, where the results are repetitively re-calculated shortly after new data is acquired) and/or in a batch-processing manner.

In certain embodiments, the portable monitoring device 50 can include a user interface 58 in order to provide information to the user and get information from the user. For example, the user interface 58 can comprise a screen (for example, liquid crystal display based or organic light-emitting diode based), and/or button(s), and/or vibration sensor (for example, piezoelectric based or based on an accelerometer or motion sensor), and/or touch sensor(s), and/or optical indicator(s), and/or vibration motor, and/or speaker, and/or microphone.

In one embodiment, the portable monitoring device 50 can include transmitter and/or receiver circuitry 64 to communicate with an external device or service or computing system (for example, see FIG. 4 and FIG. 7). For example, the portable monitoring device 50 can communicate the energy (e.g, calories) intake to an external user interface or a website (for example, www.airohealth.com). The portable monitoring device 50 can also output raw or pseudo-raw sensor data (that is, partially processed sensor data) as well as a correlation thereof. Indeed, the portable monitoring device 50 can output other nutritional or health related metrics, including any of the metrics described herein.

The portable monitoring device 50 can include transmitter and/or receiver circuitry 64 which implements or employs any form of communication link (for example, wireless, optical, or wired) and/or protocol (for example, standard or proprietary) now known or later developed, as all forms of communications protocols are intended to fall within the scope of the present invention (for example, Bluetooth, ANT (Area Network Technology), WLAN (Wireless Local Area Network), Wi-Fi, power-line networking, all types and forms of Internet based communications, and/or SMS (Short Message Service)); all forms of communications and protocols are intended to fall within the scope of the present invention.

In one embodiment, the portable monitoring device 50 makes available data (for example raw, pseudo-raw, and/or processed) to applications that run on an external device(s) (for example including third party developed or controlled applications), and/or to applications that run on a server (for example, on a webserver such as www.airohealth.com).

In one embodiment, the portable monitoring device 50 can receive data from an external device (such as a mobile phone), for example in order to modify the operation of portable monitoring device 50 (for example, improve accuracy of the calculations, and/or minimize power consumption) and/or to give feedback to user (for example, nutritional or other health related metrics, advice, instructions, and/or motivational messages) and/or to receive information from the user (for example, from an external user interface such as a mobile phone application).

In one embodiment, data intended to be sent to an external device can be stored locally (using persistent or volatile storage, not shown) if the external device cannot be reached, to be sent to the external device when the reach resumes.

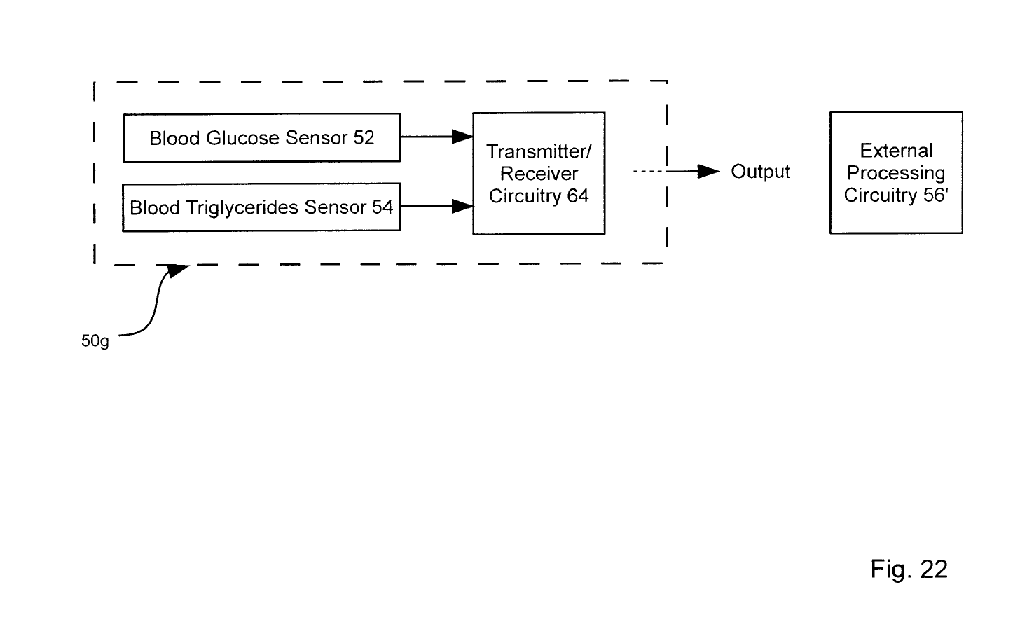

For example, in one embodiment, the portable monitoring device 50 of the present invention includes a blood glucose sensor 52 and/or a blood triglycerides sensor 54 and/or in certain embodiments other sensors such as one or more physiological or environmental sensors (for example, a motion sensor and/or a heart-rate sensor). In this embodiment, the portable monitoring device 50g however, does not include processing circuitry 56 to monitor and/or calculate energy and/or caloric intake (and/or other nutritional metrics) due to ingestion of food. In this embodiment, as shown in FIG. 22, processing circuitry 56′ is implemented “off-device” or external to the portable monitoring device 50g. Here, the portable monitoring device 10 can store (using persistent or volatile storage, not shown) and/or communicate (i) data which is representative of the blood glucose concentration and/or (ii) data which is representative of the blood triglycerides concentration to external processing circuitry 56′ (for example, on a mobile phone) wherein such external processing circuitry 56′ can monitor and/or calculate energy and/or caloric intake (and/or other nutritional metrics) due to ingestion of food of the user. Such external circuitry can implement the calculation processes and techniques in near real-time or after-the-fact. The data which is representative of the (i) blood glucose concentration and/or (ii) blood triglycerides concentration of the user can be communicated to such external processing circuitry 56′, for example, via transmitter and/or receiver circuitry 64 (see FIG. 22), removable memory, electrical or optical communication (for example, hardwired communications via USB). Importantly, such an architecture/embodiment is intended to fall within the scope of the present invention. Hybrid architectures are also contemplated whereby processing circuitry 56 is included in device 50g, however device 50g is configured so that some or all of the functions of processing circuitry 56 can be performed outside device 50 using external processing circuitry 56′.

Moreover, the portable monitoring device 50g of FIG. 22 can include all permutations and combinations of sensors (for example, one or more physiological sensor(s), and/or one or more motions sensor(s)).

In one embodiment, the portable monitoring device can implement measures to reduce power consumption, such as a change in sampling rate of the sensor(s), and/or a temporary power off of the sensor(s) and/or some or all of the processing circuitry 56 (and/or any other processing circuitry) and/or transmitter circuitry/receiver circuitry 64. For example, these power-saving techniques can be based on a time schedule (for example, cycling between being powered on for about one minute and being powered off for about four minutes), and/or based on an indicator of signal quality (for example, a motion sensor can indicate when the sensor data is most likely to be corrupted by motion artifacts, and thus could be ignored to reduce power consumption), and/or based on the user's state (for example, the nutritional sensors can be less active if it is determined that the user is sleeping).

The portable monitoring device can include a rechargeable (or non-rechargeable) battery (not shown) or ultracapacitor to provide electrical power to the circuitry and other elements of the portable monitoring device 50. In one embodiment, the energy storage element (for example, battery or storage capacitor) can obtain energy from, for example, a charger (which can be a wireless or inductive charger).

FIG. 25 is a side perspective view of an exemplary physical configuration of portable monitoring device 50 according to an embodiment and FIG. 26 is a top perspective view of an exemplary physical configuration of portable monitoring device 50 according to the same embodiment. For example, the top section can have a thickness about 6.0 mm and a width about 22.0 mm, and the bottom section can have a thickness about 7.5 mm and a width about 15 mm at the most narrow portion.

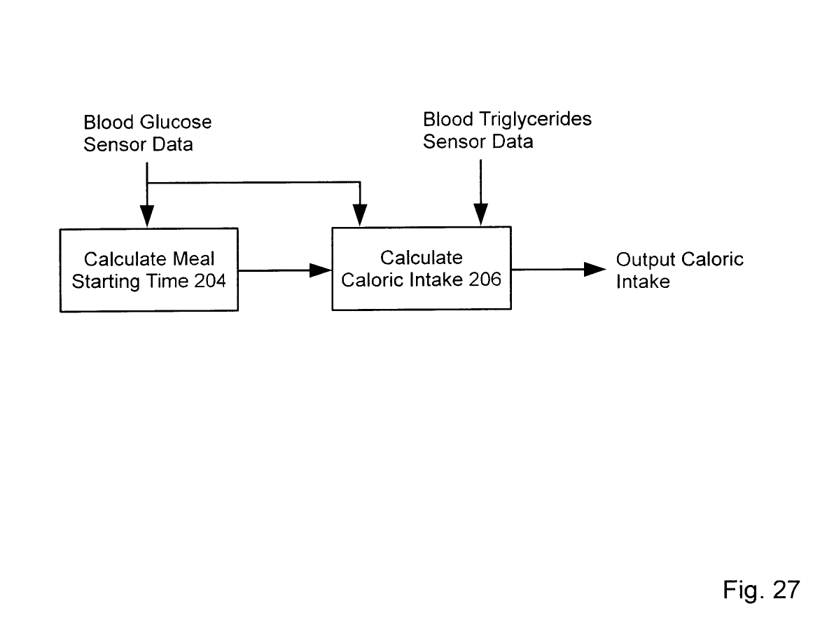

FIG. 27 is a flowchart representing an exemplary process by which processing circuitry 56 can calculate caloric intake, given data from blood glucose sensor 52 and/or data from blood triglycerides sensor 54, according to an embodiment. The process represented by FIG. 27 is equivalent to the process represented by FIG. 12, and all descriptions herein referencing FIG. 12 apply to FIG. 27 (except that block 202 is not explicitly labeled in FIG. 27).

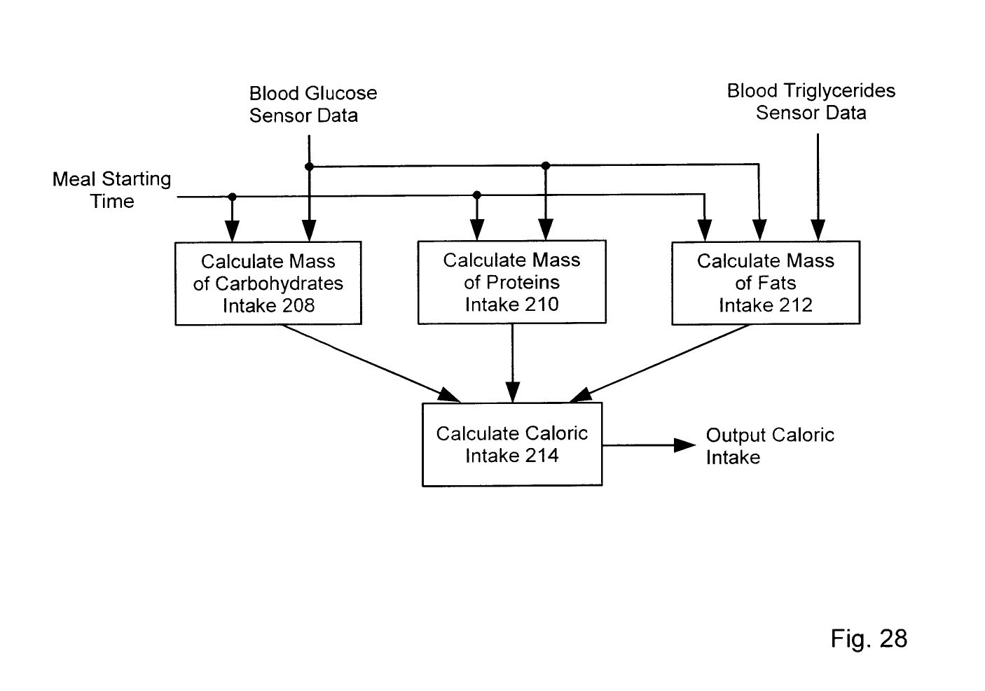

FIG. 28 is a flowchart representing an exemplary process by which processing circuitry 56 can calculate caloric intake, given data from blood glucose sensor 52 and/or data from blood triglycerides sensor 54, and the meal starting time (as calculated by block 204 of FIG. 13) according to an embodiment. The process represented by FIG. 28 is equivalent to the process represented by FIG. 13, and all descriptions herein referencing FIG. 13 apply to FIG. 28 (except that block 202 is not explicitly labeled in FIG. 28, and block 204 is not shown in FIG. 28).

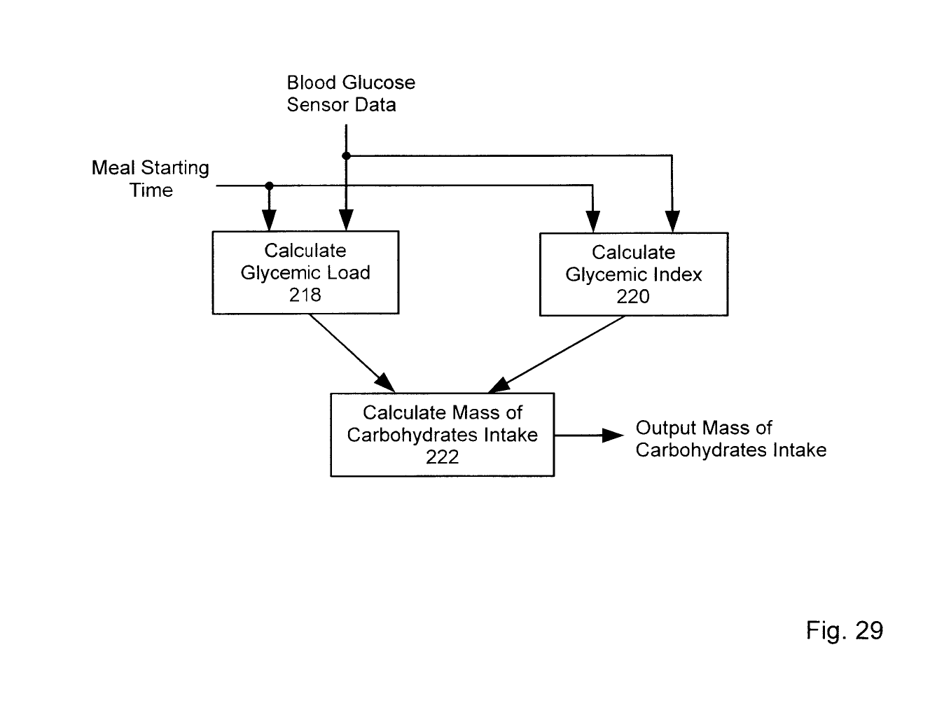

FIG. 29 is a flowchart representing an exemplary process by which processing circuitry 56 can calculate mass of carbohydrates intake, given data from blood glucose sensor 52 and the meal starting time (as calculated by block 204 of FIG. 13) according to an embodiment. The process represented by FIG. 29 is equivalent to the process represented by FIG. 16, and all descriptions herein referencing FIG. 16 apply to FIG. 29 (except that block 216 is not explicitly labeled in FIG. 29).

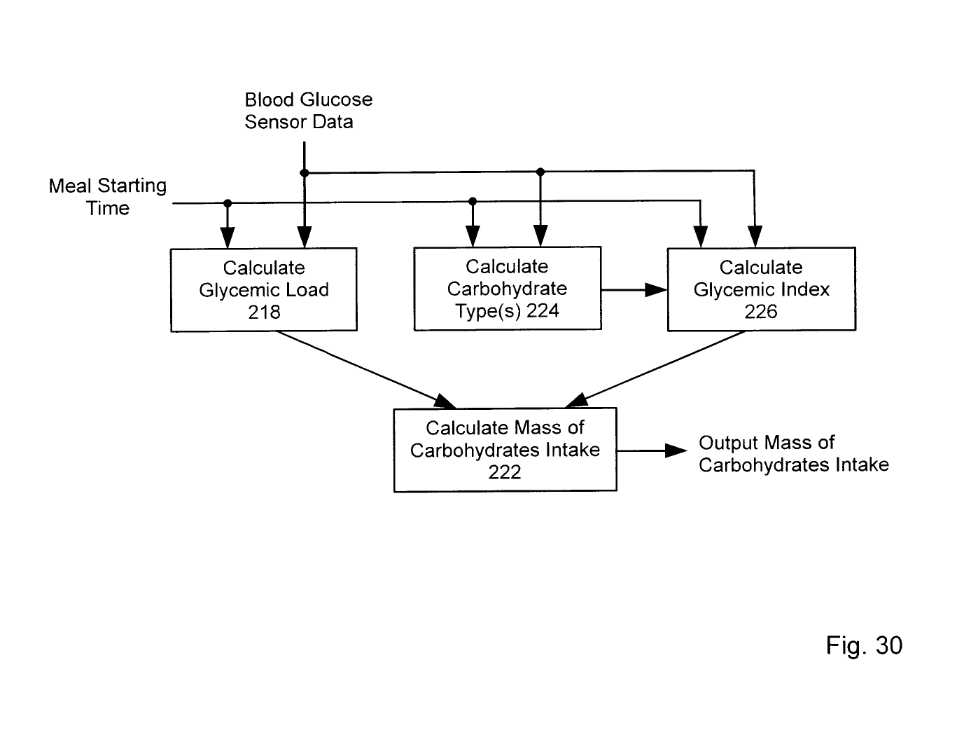

FIG. 30 is a flowchart representing an exemplary process by which processing circuitry 56 can calculate mass of carbohydrates intake, given data from blood glucose sensor 52 and the meal starting time (as calculated by block 204 of FIG. 13) according to an embodiment. The process represented by FIG. 30 is equivalent to the process represented by FIG. 17, and all descriptions herein referencing FIG. 17 apply to FIG. 30 (except that block 216 is not explicitly labeled in FIG. 30).

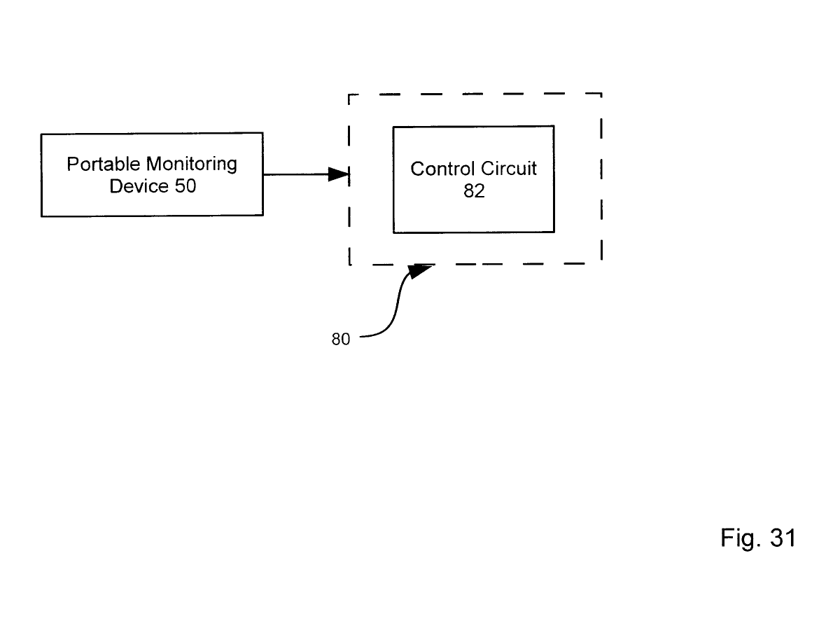

In one embodiment, with reference to FIG. 31, the portable monitoring device 50 is connected to an insulin pump 80 having a control circuit 82 being configured to meter one or more dose(s) of insulin based on the nutritional measurements provided by portable monitoring device 50. In a more particular example, the nutritional measurements can include blood glucose concentrations used by the insulin pump to meter a dose of insulin in order to reduce excessively high blood glucose concentrations and/or increase excessively low blood glucose concentrations. In another example, the nutritional measurements can include one or more of the time of the meal, the mass of carbohydrates of the meal, and glycemic index of the meal to be used by the insulin pump to meter a dose of insulin in order to counteract the anticipated effect of the meal on the blood glucose concentration. In another example, the nutritional measurements can include one or more of the time of the meal, the mass of carbohydrates of the meal, and the glycemic index of the meal to be used by the insulin pump to calculate/adjust a carbohydrates-to-insulin ratio. The carbohydrates-to-insulin ratio can be used by the insulin pump, in combination with the measured or anticipated carbohydrates of a meal, to meter a dose of insulin in order to counter the anticipated effect of the meal on the blood glucose concentration. In addition, any of the embodiments of this paragraph may be combined in an embodiment. Descriptions of an exemplary insulin pump which can be used to implement insulin pump 80 are found in Blomquist, “Carbohydrate Ratio Testing Using Frequent Blood Glucose Input,” U.S. patent application Ser. No. 11/679,712, filed Feb. 27, 2007, which is incorporated herein by reference.

In another embodiment, photoplethysmography sensor 66 can be any sensor which measures a cardiac pulse profile, for example blood pressure, blood volume, or blood flow. More specific examples include a non-contact photoplethysmography sensor, an invasive arterial blood pressure sensor, an applanation tonography sensor, or a sphygmograph sensor.

Various third party citations are made herein. The contents of each of them are incorporated herein by reference.

While the foregoing specifically discloses certain embodiments, it is to be understood that combinations, variations and subsets of those embodiments are contemplated and will now be apparent to the person skilled in the art. For example, device 50 (and its variants) can be incorporated into medical equipment for automatically administering nutrients or medications to an individual according to an individual need that is ascertainable from the calculations made by the device. A non-limiting example of such medical equipment is an insulin pump that automatically injects insulin into an individual at times and quantities that are based on measurements made by the device.

### REFERENCES

Kulcu E, Tamada J A, Reach G, Potts R O, Lesho M J. 2003. Physiological differences between interstitial glucose and blood glucose measured in human subjects. Diabetes Care 26(8): 2405-9.
Parini P, Johansson L, Bröijersén A, Angelin B, Rudling M. 2006. Lipoprotein profiles in plasma and interstitial fluid analyzed with an automated gel-filtration system. Eur J Clin Invest 36(2): 98-104.
Draper N R and Smith H. 1998. Applied regression analysis. 3rd ed. New York, N.Y.: John Wiley and Sons. 736 p.
Rifkin R M, Lippert R A. 2007. Notes on Regularized Least Squares. CSAIL, Massachusetts Institute of Technology, Cambridge, Mass.
Haykin S. 1998. Neural Networks: A Comprehensive Foundation. 2nd ed. Upper Saddle River, N.J.: Prentice Hall 842 p.
Chang C C, Lin C J. 2013. LIBSVM: A Library for Support Vector Machines. Department of Computer Science, National Taiwan University, Taipei, Taiwan.
Breiman L. 2001. Random Forests. Machine Learning 45(1): 5-32.
Wolever T M, Gibbs A L, Spolar M, Hitchner E V, Heimowitz C. 2006. Equivalent glycemic load (EGL): a method for quantifying the glycemic responses elicited by low carbohydrate foods. Nutr Metab (Lond) 3: 33.
Chlup R, Peterson K, Zapletalová J, Kudlová P, Seckar P. 2010. Extended prandial glycemic profiles of foods as assessed using continuous glucose monitoring enhance the power of the 120-minute glycemic index. J Diabetes Sci Technol 4(3): 615-24.
Moghaddam E, Vogt J A, Wolever T M. 2006. The effects of fat and protein on glycemic responses in nondiabetic humans vary with waist circumference, fasting plasma insulin, and dietary fiber intake. J Nutr 136(10): 2506-11.
Brand-Miller J C, Stockmann K, Atkinson F, Petocz P, Denyer G. 2009. Glycemic index, postprandial glycemia, and the shape of the curve in healthy subjects: analysis of a database of more than 1,000 foods. Am J Clin Nutr 89(1): 97-105.
O'Keefe J H, Gheewala N M, O'Keefe J O. 2008. Dietary strategies for improving post-prandial glucose, lipids, inflammation, and cardiovascular health. J Am Coll Cardiol 51(3): 249-55.
Petersen B L, Ward L S, Bastian E D, Jenkins A L, Campbell J, Vuksan V. 2009. A whey protein supplement decreases post-prandial glycemia. Nutr J 8: 47.

1. A device for monitoring nutritional intake comprising:
at least one biosensor for receiving at least one of blood glucose data, blood triglyceride data, and, other nutrition-related physiological data when proximate to a blood vessel;
a processing circuit connected to the biosensor output for calculating a nutritional measurement including at least a time representing a beginning of ingesting of food and a caloric intake after said time;
an output device connected to said processing circuit for outputting at least one of said time and said caloric intake.

2. The device according to claim 1 wherein said output device is further connected to an insulin pump having a control circuit; said control circuit being configured to meter a dose of insulin based on said nutritional measurement.

3. The device according to claim 1 wherein said output device comprises a display configured to generate said nutritional measurement.

4. The device according to claim 1 wherein said output device comprises a transmitter circuit for sending said nutritional measurement or said biosensor output data to an external processing circuitry

5. The device according to claim 1 wherein said processing circuit is further configured to calculate, as part of said nutritional measurement, at least one of a mass of carbohydrate intake, a mass of protein intake, a mass of fat intake, a glycemic index, and a glycemic load.

6. The device according to claim wherein said biosensor is a photoplethysmography sensor.

7. The device according to wherein 1 said caloric intake is calculated by said processing circuit according to the following:
Cᵢₙₜₐₖₑ=4*m_{carbohydrates}+4*mₚᵣₒₜₑᵢₙₛ+9*m_{fats} (1)
where Cᵢₙₜₐₖₑ is the caloric intake [kcal], m_{carbohydrates} is the mass of carbohydrates intake [grams], mₚᵣₒₜₑᵢₙₛ is the mass of proteins intake [grams], and m_{fats} is the mass of fats intake [grams].

8. The device according to claim 7 wherein said processing circuitry calculates mass of carbohydrates intake according to:
m_{carbohydrates}=GI/GL*100, (2)
where m_{carbohydrates} is the mass of carbohydrates intake [grams], GL is the glycemic load, and GI is the glycemic index.

9. The device according to claim 8 wherein said GL value is calculated by said processing circuit according to:
GL=IAUC/IAUC_{1g}, (3)
where GL is the glycemic load, IAUC is Incremental Area Under the Curve of the blood glucose concentration data, and IAUC_{1g} is the Incremental Area Under the Curve of the blood glucose concentration data due to intake of 1 gram of glucose.

10. The device according to claim 9 wherein said IAUC is calculated by said processing circuit as the area under the curve of blood glucose concentration with respect to time, wherein a baseline blood glucose concentration is subtracted, according to about a predefined period of time following the start of a meal, and wherein negative excursions relative to said baseline are excluded.

11. The device according to claim 9 wherein said predefined period of time is between about one hour and about four hours

12. The device according to claim 1 wherein said processing circuitry is configured to calculate mass of fats intake from the blood glucose concentration signal by first calculating a cross-correlation of the blood glucose concentration signal and a target signal chosen to isolate effects of fats intake on said blood glucose concentration signal.

13. The device of claim 12 wherein said mass of fats is calculated according to:
blood_glucose_{fat}[n]=(blood_glucose*fat_target[n], (4)
where blood_glucose_{fat} is the fat-correlated blood glucose signal, n is the time index, blood_glucose is the blood glucose concentration signal, fat_target is representative of the blood glucose concentration signal when fat is ingested in relative isolation, and * is a cross-correlation operator.

14. The device of claim 12 wherein said processing circuitry, given blood_glucose_{fat}, is configured to calculate mass of fats intake according to:
f=IAUC_{correlated}/IAUC_{1g} (5)
where m_{fats} is the mass of fats intake [grams], IAUC_{correlated} is the Incremental Area Under the Curve of blood_glucose_{fat}; IAUC_{1g} is the Incremental Area Under the Curve of blood_glucose_{fat} due to intake of 1 gram of fat; and the employed IAUC_{correlated} value is adjusted based on a function according to:
IAUC_{correlated}′=f(IAUC_{correlated}), (6)
where IAUC_{correlated}′ is the resulting adjusted Incremental Area Under the Curve (of blood_glucose_{fat}), f() is a polynomial function and IAUC_{correlated} is the original Incremental Area Under the Curve (of blood_glucose_{fat}).
